(19) United States
(12) Patent Application Publication
(10) Pub. No.: US 2010/0236137 A1
(43) Pub. Date: Sep. 23, 2010

(54) SYSTEMS AND METHODS FOR PRODUCING EICOSAPENTAENOIC ACID AND DOCOSAHEXAENOIC ACID FROM ALGAE

(75) Inventors: Benjamin Chiau-pin Wu, San Ramon, CA (US); David Stephen, Davis, CA (US); Gaye Elizabeth Morgenthaler, Woodside, CA (US); David Vancott Jones, Woodside, CA (US)

Correspondence Address: JONES DAY, 222 EAST 41ST ST, NEW YORK, NY, 10017, US

(73) Assignee: LiveFuels, Inc., San Carlos, CA (US)

(21) Appl. No.: 12/731,024

(22) Filed: Mar. 24, 2010

Related U.S. Application Data
(63) Continuation-in-part of application No. 12/565,612, filed on Sep. 23, 2009.
(60) Provisional application No. 61/099,503, filed on Sep. 23, 2008.

(51) Int. Cl. C12P 7/64; C11B 1/10; C10L 1/188; A01K 61/00

(52) U.S. Cl. 44/385; 554/8; 435/134; 554/1; 119/215

(57) ABSTRACT

Provided herein are systems and methods for producing eicosapentaenoic acid (EPA) and docosahexaenoic acid (DHA) and/or derivatives and/or mixtures thereof by growing algae that produce the oils containing EPA and/or DHA and/or derivatives and/or mixtures thereof, harvesting the algae with fish in one or more enclosed systems, and then processing fish to separate and purify the EPA and/or DHA. The multi-trophic systems provided herein comprise at least one enclosure that contains the algae and the fishes, and means for controllably feeding the algae to the fishes. Also provided herein are the lipid compositions extracted from the fishes.

This application is a continuation-in-part application of U.S. application Ser. No. 12/565,612, filed Sep. 23, 2009, which claims the benefit of U.S. Provisional Application No. 61/099,503, filed Sep. 23, 2008, each of which is incorporated by reference in its entirety.

### 1. INTRODUCTION

Provided herein are systems and methods for producing lipid compositions containing eicosapentaenoic acid (EPA) and/or docosahexaenoic acid (DHA) and/or derivatives and/or mixtures thereof by growing algae that produce those oils, harvesting the algae with fish, and then processing fish to separate and purify the EPA and/or DHA.

### 2. BACKGROUND OF THE INVENTION

In the early 1980s, the Inuit were found to have unusually low rates of heart disease despite their high-fat diet rich in fish. Researchers later showed that it was the omega-3 fatty acids that provided the beneficial effects, specifically the marine-derived eicosapentaenoic acid, C20:5n-3 (EPA) and docosahexaenoic acid, C22:6n-3 (DHA). In addition to beneficial effects on cardiovascular disease, EPA and DHA have since been linked to beneficial effects on arthritis, sleep disorders, high blood pressure, high cholesterol, heart arrhythmias, coronary heart disease, insomnia, depression, anorexia, schizophrenia, hypertension, and attention deficit hyperactivity disorder (ADHD). It is estimated that 84,000 deaths annually are attributable to insufficient dietary omega-3 fatty acids. Danaei, G. et al., 2009, PLoS Medicine 6(4):1-23. While still being studied, many experts believe that EPA and DHA encourage the production of body chemicals that help control inflammation.

The most widely available source of EPA and DHA is oily fish such as salmon, herring, mackerel, anchovies, and sardines. Fish oil supplements containing EPA and DHA are often made from pelagic oily fish like menhaden, sardines, and anchovies. Alarmingly, these fish resources are disappearing at a rapid pace. Leading researcher, Boris Worm, predicts that 90% of fish and shellfish used to feed people worldwide may be gone by 2048 with present trends. The global production of fish oil and fishmeal has reached a plateau over the past few years. Present methods to manufacture EPA and DHA through fermentation biotechnological processes have steadily improved, but remain too costly for most applications, except as an additive to high-priced infant formula. A new source of inexpensive EPA and DHA is critically needed.

### 3. SUMMARY OF THE INVENTION

Provided herein are inexpensive and energy-efficient methods and systems for producing EPA and/or DHA from algae. As used herein the term “EPA” refers to eicosapentaenoate, eicosapentaenoic acid, and/or derivatives thereof including, but not limited to esters, glycerides, phospholipids, sterols, and/or mixtures thereof. As used herein the term “eicosapentaenoate” refers to all-cis-5,8,11,14,17-eicosapentaenoate (Formula 1).

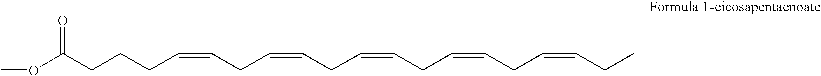

As used herein the term “eicosapentaenoic acid” refers to an eicosapentaenoate moiety in acid form, or all-cis-5,8,11,14,17-eicosapentaenoic acid (Formula 2).

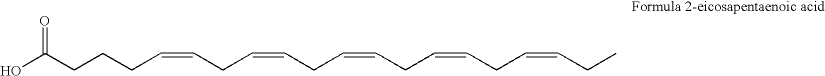

EPA in fish oil is commonly found as ethyl eicosapentaenoate (Formula 3), but EPA is also found in lipids such as acylglycerides, phospholipids, and waxy esters.

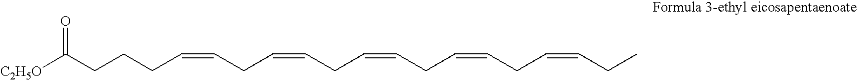

As used herein the term “DHA” refers to docosahexaenoate, docosahexaenoic acid, and/or derivatives thereof including, but not limited to esters, glycerides, phospholipids, sterols, and/or mixtures thereof. As used herein the term “docosahexaenoate” refers to all-cis-4,7,10,13,16,19-docosahexaenoate (Formula 4).

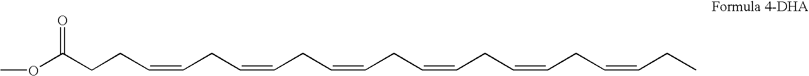

As used herein the term “docosahexaenoic acid” refers to a docosahexaenoate moiety in acid form, or all-cis-4,7,10,13,16,19-docosahexaenoic acid (Formula 5).

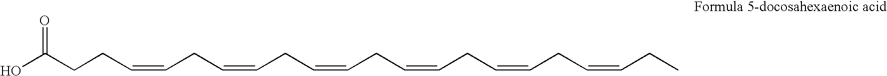

DHA in fish oil is commonly found as ethyl docosahexaenoate (Formula 6), but DHA is also found in lipids such as acylglycerides, phospholipids, and waxy esters.

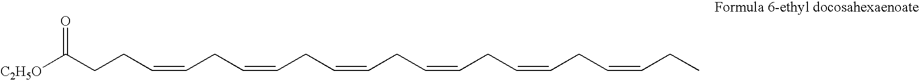

In certain embodiments, provided herein are methods that comprise cultivating autotrophic EPA- and/or DHA-producing algae (“EPA and/or DHA algae”), harvesting them with filter-feeding organisms such as fish, and then converting the fish to EPA- and/or DHA-containing oils and fishmeal using conventional reduction industry practices. In certain embodiments, methods provided herein avoid standard algae dewatering and drying steps, which can be expensive in terms of capital and energy. These steps typically require centrifugation and oil extraction with organic solvents. In certain embodiments, the methods can further comprise providing a multi-trophic system wherein the algae are controllably fed to the fish, while the fish is growing from fry to juvenile, from juvenile to adult, from fry to adult, or growing adult to larger sizes or grow-out. The harvesting step can further comprise gathering the fish when the fish has reached a fish biomass set point, determined by either mass, density, lipid content, or lipid profile.

In certain embodiments, provided herein are methods for producing an EPA and/or DHA-containing oil, said method comprising:

(i) growing one or more algae species that produce EPA and/or DHA in a first enclosed-container system or open-pond system;

(ii) harvesting said algae, comprising controllably feeding said algae to one or more zooplankton and/or fish species that feed on said algae in said first enclosed-container system or open-pond system, or in a second enclosed-container system or open-pond system, wherein said fish species feed on said algae and/or zooplankton; and

(iii) extracting EPA- and/or DHA-containing lipids from the fish, wherein said EPA- and/or DHA-containing lipids are processed to concentrate and purify EPA and/or DHA.

In one embodiment, the growing step and the harvesting step are carried out simultaneously in said first enclosed-container system or open-pond system. In one embodiment, the growing step and the harvesting step are carried out successively in said first enclosed-container system or open-pond system and said second enclosed-container system or open-pond system, respectively.

In certain embodiments, the harvesting step comprises transferring a portion of a population of the fish or the entire population of fish at least once to said second enclosed-container system or open-pond system that has a lower loading density than said first enclosed-container system or open-pond system. In one embodiment, the harvesting step comprises feeding the fish in said first or said second enclosed-container system or open-pond system that comprises the algae at a concentration of 10 to 1000 mg/L, based on a ash-free dry weight basis.

In certain embodiments provided herein are methods for making EPA- and/or DHA-containing products that can be used for human consumption (fatty acids used for nutraceuticals or other dietary supplements) or animal feeds, such as fishmeal and oil used for the growth of terrestrial, aquatic, and avian species, or boosting the EPA and/or DHA in products derived from the animals, such as milk, eggs, or meat.

In certain embodiments, the autotrophic EPA and/or DHA algae are grown in engineered enclosed systems (photobioreactors) and/or open systems (ponds or raceways). The algae cultivation may be either as monoculture species or a consortium of species depending on the location and desired product(s). In one embodiment, the algae production begins in laboratory systems at volumes ranging from 50 ml to 10 liters. The volume of the algae solution starts at typically 50-200 ml. When the algae concentration is sufficiently dense (50-1000 mg/L, based on ash-free dry weight), the algae concentrate, or inoculum, is transferred to a larger vessel that is 2 to 20 times larger. Water and nutrients are added to fill the vessel, thereby diluting the algae concentration down to 5-100 mg/L. After several days, depending on the reproduction rate of the algae, the concentration will reach densities again in the 50-1000 mg/L range, based on ash-free dry weight. Successive scale-up using this procedure would increase the algae volume up to 10-400 liters. At this stage, the algae can be fed directly to the fish, or transferred to even larger vessels, open ponds or raceways for further production. In certain embodiments, the batch production of algae concentrate described herein can also be performed continuously or semi-continuously.

In one embodiment, the algae inoculum is transferred to an open pond or raceway to help maintain the population density of the desired algae species or consortium. The continuous addition of the species or consortium would help maintain the dominant strain(s) in the pond or raceway. While the number of algae species in this open consortium is expected to be in the hundreds or thousands, in certain embodiments, the number of dominant species (>50% of total mass) can be less than 1000, less than 900, less than 800, less than 700, less than 600, less than 500, less than 400, less than 300, less than 200, less than 100, less than 90, less than 80, less than 70, less than 60, less than 50, less than 40, less than 30, less than 20, less than 10, or less than 5. Furthermore, the algae consortium can be further controlled through manipulation of the cultivation conditions, such as nutrient concentration, salinity, alkalinity, pH, temperature, CO₂ concentration, and mixing (homogeneity of the water). Functionally, these control mechanisms will affect both the rate of photosynthesis, reproduction, and predation at each trophic level independently and in concert. In another embodiment, the algae are grown as monocultures in photobioreactors (PBRs), open ponds, or raceways, and then fed directly to the fish.

In one embodiment, the growing step comprises culturing said algae in a successive scale-up system comprising:

(i) inoculating said algae in a volume of 50 to 200 ml;

(ii) transferring said algae culture to a vessel, open pond, or raceway that is 2 to 20 times larger in volume when the algae concentration reaches 50 to 1000 mg/L, wherein said transferring step is repeated until a desired amount of algae is grown; and

(iii) harvesting the algae as a single batch or semi-batch wise, wherein a fraction of the algae is harvested daily and replaced with additional water and nutrients. In one embodiment the fraction of the algae harvested is 1/10, 1/9, ⅛, 1/7, ⅙, ⅕, ¼, ⅓, ½, ⅔, ¾, ⅘, ⅚, ⅞, 8/9, or 9/10. In one embodiment, the fraction of the algae is harvested continuously.

In certain embodiments, the fish are controllably fed EPA and/or DHA algae to a predetermined ration level or to satiation. The EPA and/or DHA algae can either be the primary diet, or a supplement where the primary diet can include formulated feeds from animal or plant sources, and zooplankton (e.g., rotifers and anemia). In another embodiment, the fish are fed zooplankton that were fed EPA and/or DHA algae (“EPA and/or DHA zooplankton”). The EPA and/or DHA are synthesized by the algae and are transferred up the food chain. While de novo synthesis of EPA and/or DHA in many species has been demonstrated, the efficiencies are usually extremely low.

In certain embodiments, provided herein are at least three methods that can be employed to control the amount of EPA and/or DHA algae that the fish eat. In one embodiment, the algae and the fish are grown in the same enclosure, pond, or raceway, wherein the stocking density of the fish limits the algae available to each fish. Fish can be added or removed as the algae concentration deviates from the desired levels. In another embodiment, the algae are transported to the fish by pumps. The amount of feed is adjusted by controlling the pump rate and time. In another embodiment, the fish are allowed to swim to the algae in an adjacent enclosure, pond, or raceway. Providing the fish access to the enclosure, pond, or raceway controls the feed amount, much like limiting the accessible pasture for a grazing cow.

In certain embodiments, the feeding of the fish can continue until at least a certain proportion of the fish, e.g., 50%, grow to or exceed a predetermined biomass set point. The fish biomass set point can be determined by the weight, length, body depth, fat content, or lipid profile of the fish at a certain age, such as but not limited to 1 week, 2 weeks, 3 weeks, 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 30 months, or 36 months. In certain embodiments, wherein the fish and the algae are co-cultured in a pond or an enclosure, an algal biomass set point can be used to determine the feeding rate or the number, size, or age of fish in the enclosure.

In certain embodiments, the harvesting step comprises bringing the algae to the fish, or conversely bringing the fish to the algae, thus permitting the fish access to the algae. To ensure that the fish feed on the algae to a predetermined ration level or satiation, the concentration of algae in the fish enclosure is maintained at a level where the amount of algae that is available to the fishes is not limiting the growth of the fishes, e.g., about 10 to 1000 mg/L. In one embodiment, the harvesting step can comprise feeding the algae to a population of fishes in a first fish enclosure, and transferring a portion of the population or the entire population of fishes at least once to at least one other fish enclosure that has a lower loading density than the first fish enclosure. In another embodiment, the harvesting step may be repeated multiple times to maximize the gain in fish biomass. In another embodiment, the harvesting step can further comprise restocking the system with the algae and/or the fish periodically or continuously. In another embodiment, the harvesting step can comprise culturing the algae and the fish in an enclosure, wherein the fish feed on the algae continuously.

In certain embodiments, the methods can comprise use of any freshwater, marine, or briny species of algae and fishes. The algae of the embodiments provided herein can comprise blue-green algae, green algae, diatoms, and dinoflagellates. The algae of the embodiments provided herein can comprise species of Skeletonema, Cyanophyceae, Trichodesmium, Cryptosphaera Coelastrum, Chlorosarcina, Micractinium, Porphyridium, Nostoc, Closterium, Elakatothrix, Cyanosarcina, Trachelamonas, Euglena, Phacus, Synechocystis, Oscillatoria, Lyngbya, Kirchneriella, Carteria, Cryptomonas, Chlamydamonas, Synechococcus, Crococcus, Anacystis, Calothrix, Planktothrix, Anabaena, Hymenomonas, Isochrysis, Pavlova, Monodus, Monallanthus, Platymonas, Amphiprora, Chaetoceros, Pyramimonas, Nannochloropsis, Gymnodinium, Alexandrium, Cochlodinium, Dinophysis, Gyrodinium, Prorocentrum, Chattonella, Heterosigma, Glyphodesmis, Synedra, Neidium, Pinnularia, Stauroneis, Papiliocellulus, Scolioneis, Fallacia, Surirella, Entomoneis, Auricula, Stephanodiscus, Chroococcus, Staurastrum, Netrium, Chlorella, Amphora, Cymbella, Thalassiosira, Cylindrotheca, Rhodamonas, Nannochloropsis, Nitzchia, Pseudonitzchia, Navicula, Craticula, Gyrosigma, Pleurosigma, Melosira, Cosnodiscus, Haematococcus, Botryococcus, and/or Tetraselmis.

In certain embodiments, the harvesting methods can comprise use of planktivorous, herbivorous, or omnivorous fishes of the order Clupiformes, Siluriformes, Cypriniformes, Mugiliformes, and/or Perciformes. Preferably, at least one planktivorous species of fish in the order Clupiformes are used. Non-limiting examples of useful fishes, including menhaden, shads, herrings, sardines, hilsas, anchovies, catfishes, carps, milkfishes, shiners, paddlefish, and/or minnows.

In certain embodiments, the extraction of lipids from the fishes can comprise heating the fish to a temperature between 70° C. to 100° C., pressing the fishes to release the lipids, and collecting the lipids. Separation of the lipids from an aqueous phase and/or a solid phase can be included in the extraction step. The entire fish or a portion thereof can be used to extract lipids. If EPA and/or DHA concentrates are desired, several established methods could be employed, including chromatography, fractional or molecular distillation, enzymatic splitting, low-temperature crystallization, supercritical fluid extraction, or urea complexation.

In a preferred embodiment, a method for producing EPA and/or DHA from algae comprises: (i) providing a multi-trophic system comprising algae and a population of fish in a plurality of enclosures, wherein the enclosures are on land adjacent to a coast or in natural or artificial estuaries or in open waters; (ii) growing the algae in one or more of the plurality of enclosures; (iii) harvesting the algae by controllably feeding the algae to the population of fish, wherein at least a portion of the population of fish grows from fry to adulthood; (iv) gathering at least a portion of the population of fish; (v) extracting lipids from the gathered fish; and (vi) separating and purifying the lipids to produce the EPA and/or DHA. Preferably, the algae and the fish are indigenous to the area. In certain embodiments, algae provided herein are not grown in open waters such as rivers, streams, lakes, seas, or oceans. In certain embodiments, algae provided herein are not fed to wild or wild-caught fish.

In another embodiment, multi-trophic systems for producing EPA and/or DHA comprising algae and fish in a plurality of enclosures, means for controllably feeding the algae to the fish, means for extracting lipids from the fish, are provided. The system can further comprises means for measuring fish biomass, means for gathering the fishes, means for extracting lipids from the fishes, and means for separating the lipids into EPA and/or DHA and non-EPA and/or DHA components.

In yet another embodiment, also provided herein are products resulting from practicing the methods, such as a composition comprising lipids derived from fish that are fed with algae according to the methods provided herein.

### 4. BRIEF DESCRIPTION OF DRAWINGS

FIG. 1 illustrates an exemplary method of obtaining EPA and/or DHA from algal fed fish.

FIG. 2 illustrates an exemplary system for harvesting algae by fish and using the fish to produce EPA and/or DHA.

FIG. 3 illustrates a graph of a desirable alga due to the proportion of EPA and/or DHA present in the strain.

### 5. DETAILED DESCRIPTION OF THE INVENTION

Embodiments provided herein address the important issue that most of the oil-bearing pelagic fish, such as menhaden, anchovies, and sardines, are being rapidly overfished from our oceans. These fish are also the primary source of EPA and/or DHA, the essential fatty acids that are critical to the natural food chain and extremely beneficial to human health.

While these fish can synthesize EPA and/or DHA de novo, the efficiency can be extremely low. The EPA and/or DHA therefore originate primarily from their food, either algae, which the fish consumes either directly, or zooplankton which also eats the algae. The present embodiments provide the production of EPA and/or DHA through co-cultivation of algae and fish, with the EPA and/or DHA being produced by the algae and passed up the food chain.

While the primary source of EPA and/or DHA is wild-caught fish, Martek Biosciences Co. has patented fermentation methods using heterotrophic algae that consume sugars (see e.g., U.S. Pat. Nos. 6,750,048; 7,351,558; 7,662,598; 7,678,931). This biotechnology process requires corn syrup and expensive, sterile and highly controlled systems that are practiced in the biotechnology industry. The post-fermentation processing to separate the algae from the water, dry the algae, and extract/purify the oil further adds to the processing costs. While the fermentation process is able to produce DHA-containing, high-priced infant formulas, its exorbitant processing costs limit its application to only the most high-valued products.

Alternatively, photosynthetic algae that produce EPA and/or DHA could be cultivated in large outdoor ponds, raceways, or photobioreactors. The challenge in this approach is that the algal biomass is relatively dilute considering the volume of water. Producing a gallon of oil requires processing of about 20,000 to 40,000 gallons of water. The energy cost of transporting and processing such a large volume of water is high. As example, assuming that algae with 25% lipids can be produced at 25 g/m²/day, approximately 2,500 gallons of oil/acre/year could be produced. Remarkably, 50 million gallons of water would have to be processed to produce this oil. The standard approach of pumping water to a centralized facility for dewatering is simply too energy-intensive and cost prohibitive.

At the central processing plant, the conventional process involves separating the algae from the pond water and dewatering the algae. Typically, dewatering is accomplished by centrifugation to remove the water and evaporation of the remaining moisture by heat. Water has a high heat capacity and thus, a large amount of energy is required to evaporate the water associated with the algae. With a wet algae paste containing 15% solids, the energy required to dry the paste is essentially the amount of energy that's contained in the algae and therefore very costly.

The inventors recognize the problems with the existing fishing industry, algae fermentation, and photosynthetic algae production, and present a cost-effective and energy-efficient solution in the embodiments provided herein. The embodiments provided herein are directed to the use of fish to harvest algae and the lipids of the fish to produce EPA and/or DHA. The present embodiments also provide controlled multi-trophic systems in which algae are cultured and are consumed by planktivorous organisms, such as fishes. Algae occupy one of the lowest trophic levels in most aquatic ecological systems. Rather than harvesting the algae directly from water, the inventors take advantage of the natural order in a trophic system by collecting the energy captured by algae from planktivorous organisms that occupy a higher trophic level. Thus, instead of concentrating algae mechanically and extracting lipids from the algae, the methods provided herein employ a population of fishes and other planktivorous organisms that feed on the algae to harvest the algae. By consuming the algae, the fishes at a higher trophic level (e.g., trophic level 2) convert the algal biomass into fish biomass which comprises lipids that contain EPA and/or DHA. Because the fishes obtain a substantial part of their energy from the algae, little to no additional energy need be added to the system in order to harvest the algae. For example, adult menhaden (weighing on average 1 lb) are estimated to filter phytoplankton from seawater continuously at a rate of 7 gallons per minute (gpm) with minimal energy expenditure (Peck, J. I., 1893. On the food of the menhaden. Bull. U.S. Fish. Comm. 13: 113-126). The inventors estimate that adult menhaden requires 3-5 watts of energy when filter-feeding. In comparison, one of the largest available centrifuges (manufactured by GEA Westfalia Separator, Inc.) processes 30 gpm and consumes 18,000 watts (25 HP), or 1000-fold greater energy requirement than the menhaden at the same filtration rate. In fact, base energy expenditure of fish are typically 10-30 times lower than in mammals because of ectothermy, ammonotelism, and buoyancy (Guillaume, J., Kaushik, S., Bergot, P., and Metalller, R. Nutrition and Feeding of Fish and Crustaceans. Springer Publishing, 2001). The fish is a natural concentrator and harvester of algae and a one-pound menhaden contains as much energy as 800 gallons of algae-containing water, but requires significantly less energy to process than that amount of water, resulting in a larger net energy gain.

Many fishes feed on algae as well as zooplankton and/or detritus. In fact, most planktivorous fish preferentially feed on zooplankton which tends to limit the predation of algae by zooplankton, a frequent cause of algae population crashes. Such fishes can potentially recover EPA and/or DHA present in detritus, or lost to zooplankton that graze on phytoplankton. Transgenic fish and genetically improved fish that possess a higher growth rate or higher capacity of producing and/or accumulating EPA and/or DHA on a diet of algae, can be used in the harvesting methods provided herein. In addition, piscivorous fishes (e.g., at trophic level 3) can also be used in the system to harvest fishes of a lower trophic level, such as the herbivorous, planktivorous, and detritivorus fishes. High value piscivorous fishes can be sold as food for human consumption and provides an additional revenue.

The extraction of lipids from fish is a step in the commercial process for producing fish meal and fish oil. Because harvesting and processing the fishes do not require removing and heating large volumes of water, as practiced in conventional algae cultivation, an enormous energy cost savings can be realized. The capital and energy cost expended in processing fish is more favorable than directly processing algae.

Certain embodiments provided herein are distinguishable from the seafood industry in several aspects. Historically, fish oil and fish wastes had been disposed of by burning as fuel at the smaller scale. However, the fish oil and fish waste generated by the seafood industry were obtained from wild fish that had not been raised on cultured algae. There is considerable diversity in the age and the types of fish that are captured from wild stocks that feed in open water. Unlike the embodiments provided herein, the composition and yield of lipids from captured fish are variable and highly unpredictable, and thus they are not reliable sources of EPA and/or DHA. The fish used in the embodiments provided herein are cultured in an enclosure, and gathered when the population has reached a certain average biomass set point or when the enclosure has reached its loading capacity. The population of fish used in making EPA and/or DHA of the embodiments provided herein is cultured, and thus different from wild population of fish that are captured and processed by the seafood industry. The extraction of fish lipids from captured wild fish is an unsustainable practice and is not a part of the embodiments provided herein.

Moreover, farm-raised fish are known to possess an earthy or metallic off-flavor if they are processed immediately after retrieval from the enclosure in which they are cultured. In certain embodiments, to prevent development of the flavor or to remove the flavor, prior to harvesting, the farmed fish are transferred to and cultured in a clean pond that contains relatively few algae and bacteria, for a short period, such as about 7 to 14 days. During this period, the fish are not fed with the same feed (which may include algae) as before. Since the taste of the fish used in the present embodiments is unimportant, the methods provided herein need not include performing this separate culturing step in water that contains a lower algae and bacteria count than the enclosure in which the fish were cultured.

Algae inhabit all types of aquatic environments, including but not limited to freshwater, marine, and brackish environments, in all climatic regions, such as tropical, subtropical, temperate, and polar. Accordingly, certain embodiments provide controlled multi-trophic systems for culturing algae and fishes in any of such aquatic environments and climatic regions. Certain embodiments provided herein can be practiced in many geographic areas, such as but not limited to bodies of water on land, such as but not limited to, lakes, ponds, coastal land, land adjacent to rivers and bodies of water. Certain embodiments provided herein can be practiced in many parts of the world, such as the coasts, the coastal land, the contiguous zones, the territorial zones, and the exclusive economic zones of the United States. For example, a system provided herein can be established on coastal land at the coasts of Gulf of Mexico, or in the waters of the Gulf of Mexico basin, Northeast Gulf of Mexico, South Florida Continental Shelf and Slope, Campeche Bank, Bay of Campeche, Western Gulf of Mexico, and Northwest Gulf of Mexico.

For clarity of disclosure, and not by way of limitation, a detailed description of the present embodiments is divided into the subsections which follow. The algae and fishes that are useful in the methods provided herein are described in detail in Section 5.1 and 5.2 respectively. The systems and methods of harvesting algae are described in detail in Section 5.3. EPA, DHA, and other lipids of the present embodiments are described in Section 5.4.

As used herein, “a” or “an” means at least one, unless clearly indicated otherwise. The term “about,” as used herein, unless otherwise indicated, refers to a value that is no more than 20% above or below the value being modified by the term. Technical and scientific terms used herein have the meanings commonly understood by one of ordinary skill in the art to which the present embodiments pertain, unless otherwise defined.

Technical and scientific terms used herein have the meanings commonly understood by one of ordinary skill in the art to which the present embodiments pertain, unless otherwise defined. Reference is made herein to various equipment, technologies and methodologies known to those of skill in the art. Publications and other materials setting forth such known equipment, technologies and methodologies to which reference is made are incorporated herein by reference in their entireties as though set forth in full. The practice of the embodiments provided herein will employ, unless otherwise indicated, equipment, methodologies and techniques of chemical engineering, biology, ecology, and the fishery and aquaculture industries, which are within the skill of the art. Such equipment, technologies and methodologies are explained fully in the literature, e.g., Aquaculture Engineering, Odd-Ivar Lekang, 2007, Blackwell Publishing Ltd.; Handbook of Microalgal Culture, edited by Amos Richmond, 2004, Blackwell Science; Microalgae Biotechnology and Microbiology, E.W. Becker, 1994, Cambridge University Press; Limnology: Lake and River Ecosystems, Robert G. Wetzel, 2001, Academic Press; and Aquaculture. Farming Aquatic Animals and Plants, Editors: John S. Lucas and Paul C. Southgate, Blackwell Publishing, (2003), each of which are incorporated by reference in their entireties.

5.1 Algae

As used herein the term “algae” refers to any organisms with chlorophyll and a thallus not differentiated into roots, stems and leaves, and encompasses prokaryotic and eukaryotic organisms that are photoautotrophic or photoauxotrophic. The term “algae” includes macroalgae (commonly known as seaweed) and microalgae. In certain embodiments, algae that are not macroalgae are preferred. The terms “microalgae” and “phytoplankton,” used interchangeably herein, refer to any microscopic algae, photoautotrophic or photoauxotrophic eukaryotes (such as, protozoa), photoautotrophic or photoauxotrophic prokaryotes, and cyanobacteria (commonly referred to as blue-green algae and formerly classified as Cyanophyceae). The use of the term “algal” also relates to microalgae and thus encompasses the meaning of “microalgal.” The term “algal composition” refers to any composition that comprises algae, such as an aquatic composition, and is not limited to the body of water or the culture in which the algae are cultivated. An algal composition can be an algal culture, a concentrated algal culture, or a dewatered mass of algae, and can be in a liquid, semi-solid, or solid form. A non-liquid algal composition can be described in terms of moisture level or percentage weight of the solids. An “algal culture” is an algal composition that comprises live algae.

The microalgae of the embodiments provided herein are also encompassed by the term “plankton” which includes phytoplankton, zooplankton and bacterioplankton. In certain embodiments, an algal composition or a body of water comprising algae that is substantially depleted of zooplankton is preferred since many zooplankton consume phytoplankton. However, it is contemplated that many aspects of the embodiments provided herein can be practiced with a planktonic composition, without isolation of the phytoplankton, or removal of the zooplankton or other non-algal planktonic organisms. The methods of the embodiments provided herein can be used with a composition comprising plankton, or a body of water comprising plankton.

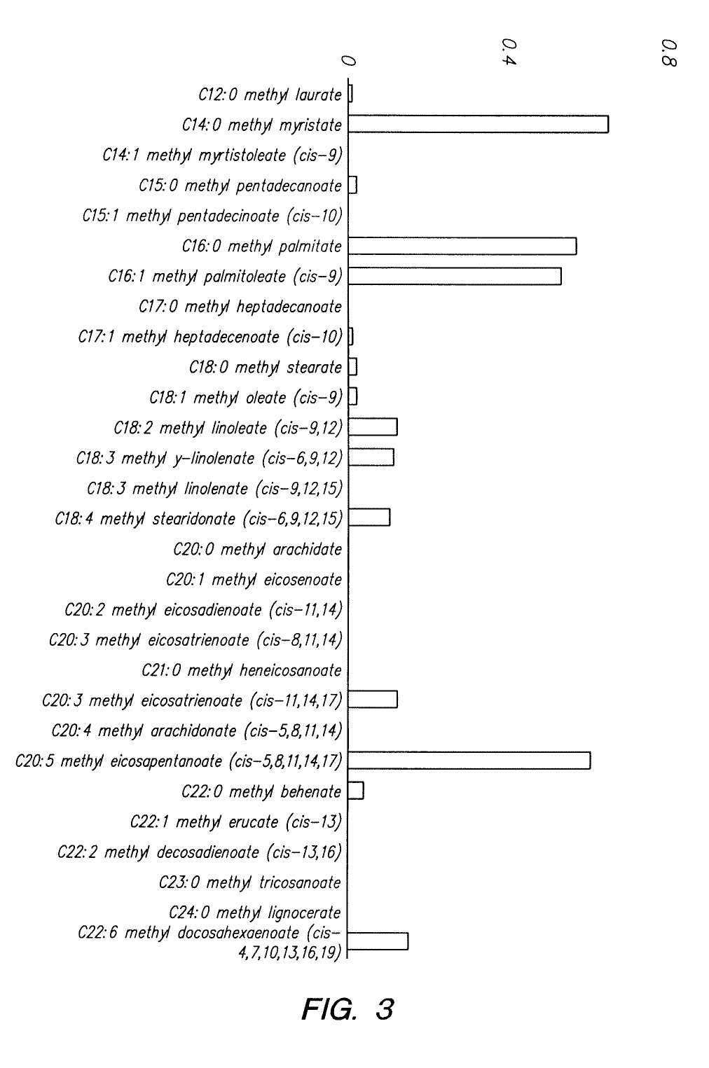

The algae of the embodiments provided herein can be a naturally occurring species, a genetically selected strain, a genetically manipulated strain, a transgenic strain, or a synthetic algae. Preferably, the algae bears at least one beneficial trait, such as but not limited to, increased growth rate, lipid accumulation, favorable lipid composition, adaptation to the culture environment, and robustness in changing environmental conditions. It is desirable that the algae accumulate excess lipids and/or hydrocarbons. FIG. 3 provides a graph of a desirable alga due to the proportion of EPA and/or DHA present in the strain.

However, lipid accumulation in the algae is not a requirement because the algal biomass, without excess lipids, can also be converted to lipids metabolically by the harvesting fish. The algae in an algal composition of the embodiments provided herein may not all be cultivable under laboratory conditions. It is not required that all the algae in an algal composition of the embodiments provided herein be taxonomically classified or characterized in order to for the composition be used in the present embodiments. Algal compositions, including algal cultures, can be distinguished by the relative proportions of taxonomic groups that are present.

The algae of the embodiments provided herein use light as their energy source. The algae can be grown under the sunlight or artificial light. In addition to using mass per unit volume (such as mg/L or g/L), chlorophyll a is a commonly used indicator of algal biomass. However, it is subjected to variability of cellular chlorophyll content (0.1 to 9.7% of fresh algal weight) depending on algal species. An estimated biomass value can be calibrated based on the chlorophyll content of the dominant species within a population. Published correlation of chlorophyll a concentration and biomass value can be used in the embodiments provided herein. Generally, chlorophyll a concentration is to be measured within the euphotic zone of a body of water. The euphotic zone is the depth at which the light intensity of the photosynthetically active spectrum (400-700 nm) exceeds 1% of the surface light intensity.

Depending on the latitude of a site, algae obtained from tropical, subtropical, temperate, polar or other climatic regions are used in the embodiments provided herein. Endemic or indigenous algal species are generally preferred over introduced species where an open culturing system is used. Endemic or indigenous algae may be enriched or isolated from local water samples obtained at or near the site of the system. It is advantageous to use algae and fishes from a local aquatic trophic system in the methods of the embodiments provided herein. Algae, including microalgae, inhabit many types of aquatic environments, including but not limited to freshwater (less than about 0.5 parts per thousand (ppt) salts), brackish (about 0.5 to about 31 ppt salts), marine (about 31 to about 38 ppt salts), and briny (greater than about 38 ppt salts) environments. Any of such aquatic environments, freshwater species, marine species, and/or species that thrive in varying and/or intermediate salinities or nutrient levels, can be used in the embodiments provided herein. The algae in an algal composition of the embodiments provided herein can be obtained initially from environmental samples of natural or man-made environments, and may contain a mixture of prokaryotic and eukaryotic organisms, wherein some of the species may be unidentified. Freshwater filtrates from rivers, lakes; seawater filtrates from coastal areas, oceans; water in hot springs or thermal vents; and lake, marine, or estuarine sediments, can be used to source the algae. The samples may also be collected from local or remote bodies of water, including surface as well as subterranean water.

One or more species of algae are present in the algal composition of the embodiments provided herein. In one embodiment, the algal composition is a monoculture, wherein only one species of algae is grown. However, in many open culturing systems, it may be difficult to avoid the presence of other algae species in the water. The inventors believe that an algae consortium can be more productive and healthier than a monoculture. In certain embodiments, a monoculture may comprise about 0.1% to 2% cells of algae species other than the intended species, i.e., up to 98% to 99.9% of the algal cells in a monoculture are of one species. In certain embodiments, the algal composition comprises an isolated species of algae, such as an axenic culture. In another embodiment, the algal composition is a mixed culture that comprises more than one species (“consortium”) of algae, i.e., the algal culture is not a monoculture. Such a culture can be prepared by mixing different algal cultures or axenic cultures. In certain embodiments, the algal composition can also comprise zooplankton, bacterioplankton, and/or other planktonic organisms. In certain embodiments, an algal composition comprising a combination of different batches of algal cultures is used. The algal composition can be prepared by mixing a plurality of different algal cultures. The different taxonomic groups of algae can be present in defined proportions. The combination and proportion of different algae in an algal composition can be designed or adjusted to enhance the growth and/or accumulation of lipids of certain groups or species of fish. In certain embodiments, microalgal composition provided herein can comprise predominantly microalgae of a selected size range, such as but not limited to, below 2000 μm, about 200 to 2000 μm, above 200 μm, below 200 μm, about 20 to 2000 μm, about 20 to 200 μm, above 20 μm, below 20 μm, about 2 to 20 μm, about 2 to 200 μm, about 2 to 2000 μm, below 2 μm, about 0.2 to 20 μm, about 0.2 to 2 μm or below 0.2 μm.

In one embodiment, a mixed algal composition can comprise one or several dominant species of macroalgae and/or microalgae. Microalgal species can be identified by DNA sequencing or microscopy and enumerated by counting visually or optically, or by techniques such as but not limited to spectroscopy, microfluidics and flow cytometry, which are well known in the art. A dominant species is one that ranks high in the number of algal cells, e.g., the top one to five species with the highest number of cells relative to other species. Microalgae occur in unicellular, filamentous, or colonial forms. The number of algal cells can be estimated by counting the number of colonies or filaments. Alternatively, the dominant species can be determined by ranking the number of cells, colonies and/or filaments. This scheme of counting may be preferred in mixed cultures where different forms are present and the number of cells in a colony or filament is difficult to discern. In a mixed algal composition, the one or several dominant algae species may constitute greater than about 10%, about 20%, about 30%, about 40%, about 50%, about 60%, about 70%, about 80%, about 90%, about 95%, about 97%, about 98%, or about 99% of the algae present in the culture. In certain mixed algal composition, several dominant algae species may each independently constitute greater than about 10%, about 20%, about 30%, about 40%, about 50%, about 60%, about 70%, about 80%, about 90%, or about 95% of the algae present in the culture. Many other minor species of algae may also be present in such composition but they may constitute in aggregate less than about 50%, about 40%, about 30%, about 20%, about 10%, or about 5% of the algae present. In various embodiments, one, two, three, four, or five dominant species of algae are present in an algal composition. Accordingly, a mixed algal culture or an algal composition can be described and distinguished from other cultures or compositions by the dominant species of algae present. An algal composition can be further described by the percentages of cells that are of dominant species relative to minor species, or the percentages of each of the dominant species. In certain embodiments, the identification of dominant species can also be limited to species within a certain size class, e.g., below 2000 μm, about 200 to 2000 μm, above 200 below 200 μm, about 20 to 2000 μm, about 20 to 200 μm, above 20 μm, below 20 μm, about 2 to 20 μm, about 2 to 200 μm, about 2 to 2000 μm, below 2 μm, about 0.2 to 20 μm, about 0.2 to 2 μm or below 0.2 μm. It is to be understood that mixed algal cultures or compositions having the same genus or species of algae may be different by virtue of the relative abundance of the various genus and/or species that are present.

It is contemplated that many different algal cultures or bodies of water which comprise plankton, can be harvested efficiently by the methods provided herein. Microalgae are preferably used in certain embodiments; while macroalgae are less preferred in certain embodiments. In specific embodiments, algae of a particular taxonomic group, e.g., a particular genera or species, may be less preferred in a culture. Such algae, including one or more that are listed below, may be specifically excluded as a dominant species in a culture or composition. However, it should also be understood that in certain embodiments, such algae may be present as a contaminant, a non-dominant group or a minor species, especially in an open system. Such algae may be present in negligible numbers, or substantially diluted given the volume of the culture or composition. The presence of such algal genera or species in a culture, composition or a body of water is distinguishable from cultures, composition or bodies of water where such algal genus or species are dominant, or constitute the bulk of the algae. The composition of an algal culture or a body of water in an open culturing system is expected to change according to the four seasons, for example, the dominant species in one season may not be dominant in another season. An algal culture at a particular geographic location or a range of latitudes can therefore be more specifically described by season, i.e., spring composition, summer composition, fall composition, and winter composition; or by any one or more calendar months, such as but not limited to, from about December to about February, or from about May to about September. The species composition of an algal culture or a body of water in an open culturing system can also be modified by changing the chemical composition of the water, including but not limited to, nutrient concentrations (N/P/Si), pH, alkalinity, and salinity. The degree of mixing in the pond can also used to control the algae consortium. Given the remarkable specialization of algae species to environmental conditions, the dominant species can vary diurnally, seasonally, and even within a pond.

In certain embodiments, one or more species of algae belonging to the following phyla can be harvested by the systems and methods provided herein: Cyanobacteria, Cyanophyta, Prochlorophyta, Rhodophyta, Glaucophyta, Chlorophyta, Dinophyta, Cryptophyta, Chrysophyta, Prymnesiophyta (Haptophyta), Bacillariophyta, Xanthophyta, Eustigmatophyta, Rhaphidophyta, and Phaeophyta. In certain embodiments, algae in multicellular or filamentous forms, such as seaweeds and/or macroalgae, many of which belong to the phyla Phaeophyta or Rhodophyta, are less preferred.

In certain embodiments, the algal composition comprises cyanobacteria (also known as blue-green algae) from one or more of the following taxonomic groups: Chroococcales, Nostocales, Oscillatoriales, Pseudanabaenales, Synechococcales, and Synechococcophycideae. Non-limiting examples include Gleocapsa, Pseudoanabaena, Oscillatoria, Microcystis, Synechococcus and Arthrospira species.

In certain embodiments, the algal composition comprises algae from one or more of the following taxonomic classes: Euglenophyceae, Dinophyceae, and Ebriophyceae. Non-limiting examples include Euglena species and the freshwater or marine dinoflagellates.

In certain embodiments, the algal composition comprises green algae from one or more of the following taxonomic classes: Micromonadophyceae, Charophyceae, Ulvophyceae and Chlorophyceae. Non-limiting examples include species of Borodinella, Chlorella (e.g., C. ellipsoidea), Chlamydomonas, Dunaliella (e.g., D. salina, D. bardawil), Franceia, Haematococcus, Oocystis (e.g., O. parva, O. pustilla), Scenedesmus, Stichococcus, Ankistrodesmus (e.g., A. falcatus), Chlorococcum, Monoraphidium, Nannochloris and Botryococcus (e.g., B. braunii). In certain embodiments, Chlamydomonas reinhardtii are less preferred.

In certain embodiments, the algal composition comprises golden-brown algae from one or more of the following taxonomic classes: Chrysophyceae and Synurophyceae. Non-limiting examples include Boekelovia species (e.g. B. hooglandii) and Ochromonas species.

In certain embodiments, the algal composition comprises freshwater, brackish, or marine diatoms from one or more of the following taxonomic classes: Bacillariophyceae, Coscinodiscophyceae, and Fragilariophyceae. Preferably, the diatoms are photoautotrophic or auxotrophic. Non-limiting examples include Achnanthes (e.g., A. orientalis), Amphora (e.g., A. coffeiformis strains, A. delicatissima), Amphiprora (e.g., A. hyaline), Amphipleura, Chaetoceros (e.g., C. muelleri, C. gracilis), Caloneis, Camphylodiscus, Cyclotella (e.g., C. cryptica, C. meneghiniana), Cricosphaera, Cymbella, Diploneis, Entomoneis, Fragilaria, Hantschia, Gyrosigma, Melosira, Navicula (e.g., N. acceptata, N. biskanterae, N. pseudotenelloides, N. saprophila), Nitzschia (e.g., N. dissipata, N. communis, N. inconspicua, N. pusilla strains, N. microcephala, N. intermedia, N. hantzschiana, N. alexandrina, N. quadrangula), Phaeodactylum (e.g., P. tricornutum), Pleurosigma, Pleurochrysis (e.g., P. carterae, P. dentata), Selenastrum, Skeletonema, Surirella and Thalassiosira (e.g., T. weissflogii).

In certain embodiments, the algal composition comprises planktons including microalgae that are characteristically small with a diameter in the range of 1 to 10 μm, or 2 to 4 μm. Many of such algae are members of Eustigmatophyta, such as but not limited to Nannochloropsis species (e.g. N. salina).

In certain embodiments, the algal composition comprises one or more algae from the following groups: Coelastrum, Chlorosarcina, Micractinium, Porphyridium, Nostoc, Closterium, Elakatothrix, Cyanosarcina, Trachelamonas, Kirchneriella, Carteria, Crytomonas, Chlamydamonas, Planktothrix, Anabaena, Hymenomonas, Isochrysis, Pavlova, Monodus, Monallanthus, Platymonas, Amphiprora, Chatioceros, Pyramimonas, Stephanodiscus, Chroococcus, Staurastrum, Netrium, and Tetraselmis.

In certain embodiments, any of the above-mentioned genus and species of algae may each be less preferred independently as a dominant species in, or be excluded from, an algal composition provided herein.

5.2 Fish

As used herein, the term fish refers to a member or a group of the following classes: Actinopteryii (i.e., ray-finned fish) which includes the division Teleosteri (also known as the teleosts), Chondrichytes (e.g., cartilaginous fish), Myxini (e.g., hagfish), Cephalospidomorphi (e.g., lampreys), and Sarcopteryii (e.g., coelacanths). The teleosts comprise at least 38 orders, 426 families, and 4064 genera. Some teleost families are large, such as Cyprinidae, Gobiidae, Cichlidae, Characidae, Loricariidae, Balitoridae, Serranidae, Labridae, and Scorpaenidae. In many embodiments, bony fishes, such as the teleosts, and/or cartilaginous fishes are used. When referring to a plurality of organisms, the term “fish” is used interchangeably with the term “fishes” regardless of whether one or more than one species are present, unless clearly indicated otherwise.

Stocks of fish used in the embodiments provided herein can be obtained initially from fish hatcheries or collected from the wild. Preferably, cultured or farmed fishes are used. The fishes may be fish fry, juveniles, fingerlings, or adult/mature fish. In certain embodiments, fry and/or juveniles that have metamorphosed are used. By “fry” it is meant a recently hatched fish that has fully absorbed its yolk sac, while by “juvenile” or “fingerling,” it is meant a fish that has not recently hatched but is not yet an adult. In certain embodiments, the fishes may reproduce in an enclosure comprising algae within the system and not necessarily in a fish hatchery. Any fish aquaculture techniques known in the art can be used to stock, maintain, reproduce, and gather the fishes used in the present embodiments.

One or more species of fish can be used to harvest the algae from an algal composition. In one embodiment, the population of fish comprises only one species of fish. In another embodiment, the fish population is mixed and thus comprises one or several major species of fish. A major species is one that ranks high in the head count, e.g., the top one to five species with the highest head count relative to other species. The one or several major fish species may constitute greater than about 10%, about 20%, about 30%, about 40%, about 50%, about 60%, about 70%, about 75%, about 80%, about 90%, about 95%, about 97%, about 98% of the fish present in the population. In certain embodiments, several major fish species may each constitute greater than about 10%, about 20%, about 30%, about 40%, about 50%, about 60%, about 70%, or about 80% of the fish present in the population. In various embodiments, one, two, three, four, five major species of fish are present in a population of fishes. Accordingly, a mixed fish population can be described and distinguished from other populations by the major species of fish present. The population can be further described by the percentages of the major and minor species, or the percentages of each of the major species. It is to be understood that in a body of water comprising a mixed fish population having the same genus or species of fish as another body of water may be different by virtue of the relative abundance of the various genera and/or species of fish present.

Fish inhabit most types of aquatic environments, including but not limited to freshwater, brackish, marine, and briny environments. As the present embodiments can be practiced in any of such aquatic environments, any freshwater species, stenohaline species, euryhaline species, marine species, species that grow in brine, and/or species that thrive in varying and/or intermediate salinities, can be used. Depending on the latitude of the system, fishes from tropical, subtropical, temperate, polar, and/or other climatic regions can be used. For example, fishes that live within the following temperature ranges can be used: below 10° C., 9° C. to 18° C., 15° C. to 25° C., 20° C. to 32° C. In one embodiment, fishes indigenous to the region at which the methods of the present embodiments are practiced, are used. Preferably, fishes from the same climatic region, same salinity environment, or same ecosystem, as the algae are used. The algae and the fishes are preferably derived from a naturally occurring trophic system.

In an aquatic ecosystem, fish occupies various trophic levels. Depending on diet, fish are classified generally as piscivores (carnivores), herbivores, planktivores, detritivores, and omnivores. The classification is based on observing the major types of food consumed by fish and its related adaptation to the diet. For example, many species of planktivores develop specialized anatomical structures to enable filter feeding, e.g., gill rakers and gill lamellae. Generally, the size of such filtering structures relative to the dimensions of plankton, including microalgae, affects the diet of a planktivore. Fish having more closing spaced gill rakers with specialized secondary structures to form a sieve are typically phytoplanktivores. Others having widely spaced gill rakers with secondary barbs are generally zooplanktivores. In the case of piscivores, the gill rakers are generally reduced to barbs. Herbivores generally feed on macroalgae and other aquatic vascular plants. Gut content analysis can determine the diet of an organism used in the present embodiments. Techniques for analysis of gut content of fish are known in the art. As used herein, a planktivore is a phytoplanktivore if a population of the planktivore, reared in water with non-limiting quantities of phytoplankton and zooplankton, has on average more phytoplankton than zooplankton in the gut, for example, greater than 50%, 60%, 70%, 80%, or 90%. Under similar conditions, a planktivore is a zooplantivore if the population of the planktivore has on average more zooplankton than phytoplankton in the gut, for example, greater than 50%, 60%, 70%, 80%, or 90%. Certain fish can consume a broad range of food or can adapt to a diet offered by the environment. Accordingly, it is preferable that the fish are cultured in a system provided herein before undergoing a gut content analysis.

Fishes that are used in the methods provided herein feed on algae, but it is not required that they feed exclusively on microalgae, i.e., they can be herbivores, omnivores, planktivores, phytoplanktivores, zooplanktivores, or generally filter feeders, including pelagic filter feeders and benthic filter feeders. In some embodiments, the population of fish useful for harvesting algae comprises predominantly planktivores. In some embodiments, the population of fish useful for harvesting algae comprises predominantly omnivores. In certain embodiments, one or several major species in the fish population are planktivores or phytoplanktivores. In certain mixed fish population of the embodiments, planktivores and omnivores are both present. In certain other mixed fish population, in addition to planktivores, herbivores and/or detritivores are also present. In certain embodiments, piscivores are used in a mixed fish population to harvest other fishes. In certain embodiments, piscivores are less preferred or excluded from the systems provided herein. The predominance of one type of fish as defined by their trophic behavior over another type in a population of fishes can be defined by percentage head count as described above for describing major fish species in a population (e.g., 90% phytoplanktivores, 10% omnivores).

The choice of fish for use in the harvesting methods provided herein depends on a number of factors, such as the palatability and nutritional value of the cultured algae as food for the fishes, the lipid composition and content of the fish, the feed conversion ratio, the fish growth rate, and the environmental requirements that encourages feeding and growth of the fish. For example, it is preferable that the selected fishes will feed on the cultured algae until satiation, and convert the algal biomass into fish biomass rapidly and efficiently. Gut content analysis can reveal the dimensions of the plankton ingested by a planktivore and the preference of the planktivore for certain species of algae. Knowing the average dimensions of ingested plankton, the preference and efficiency of a planktivore towards a certain size class of plankton can be determined. Based on size preference and/or species preference of the fishes, a planktivore can be selected to match the size and/or species of algae in the algal composition. To reduce the need to change water when an algae composition is brought to the fish in an enclosure, the algae and fish are preferably adapted to grow in a similar salinity environment. The use of matched fish and algae species in the methods provided herein can improve harvesting efficiency. It may also be preferable to deploy combinations of algae and fishes that are parts of a naturally occurring trophic system. Many trophic systems are known in the art and can be used to guide the selection of algae and fishes for use in the present embodiments. The population of fishes can be self-sustaining and does not require extensive fish husbandry efforts to promote reproduction and to rear the juveniles.

Currently, many species of fishes are farmed or captured for human consumption, making animal feed, including aquaculture feed, and a variety of other oleochemical-derived products, such as paints, linoleum, lubricants, soap, insecticides, and cosmetics. The methods provided herein can employ such species of fishes that are otherwise used as human food, animal feed, or oleochemical feedstocks, for making EPA and/or DHA. Depending on the economics of operating an algal culture facility, some of the fishes used in the present method can be sold as human food, animal feed or oleochemical feedstock. In certain embodiments, the fishes used in the present embodiments are not suitable for making animal feed, human food, or oleochemical feedstock.

It should be understood that, in various embodiments, fishes within a taxonomic group, such as a family or a genus, could be used interchangeably in various methods provided herein. The embodiments provided herein are described below using common names of fish groups and fishes, as well as the scientific names of exemplary species. Databases, such as FishBase by Froese, R. and D. Pauly (Ed.), World Wide Web electronic publication, www.fishbase.org, version (June 2008), provide additional useful fish species within each of the taxonomic groups that are useful in the present embodiments. It is contemplated that one of ordinary skill in art could, consistent with the scope of the present embodiments, use the databases to specify other species within each of the described taxonomic groups for use in the methods provided herein.

In certain embodiments, the fish population comprises fishes in the order Acipeneriformes, such as but not limited to, sturgeons (trophic level 3), e.g., Acipenser species, Huso huso, and paddlefishes (plankton-feeder), e.g., Psephurus gladius, Polyodon spathula, and Pseudamia zonata.

In certain embodiments, the fishes used in the embodiments comprise fishes in the order Clupeiformes, i.e. the clupeids, which include the following families: Chirocentridae, Clupeidae (menhadens, shads, herrings, sardines, hilsa), Denticipitidae, and Engraulidae (anchovies). Exemplary members within the order Clupeiformes include but are not limited to, the menhadens (Brevoortia species), e.g, Ethmidium maculatum, Brevoortia aurea, Brevoortia gunteri, Brevoortia smithi, Brevoortia pectinata, Gulf menhaden (Brevoortia patronus), and Atlantic menhaden (Brevoortia tyrannus); the shads, e.g., Alosa alosa, Alosa alabamae, Alosa fallax, Alosa mediocris, Alosa sapidissima, Alos pseudoharengus, Alosa chrysochloris, Dorosoma petenense; the herrings, e.g., Etrumeus teres, Harengula thrissina, Pacific herring (Clupea pallasii pallasii), Alosa aestivalis, Ilisha africana, Ilisha elongata, Ilisha megaloptera, Ilisha melastoma, Ilisha pristigastroides, Pellona ditchela, Opisthopterus tardoore, Nematalosa come, Alosa aestivalis, Alosa chrysochloris, freshwater herring (Alosa pseudoharengus), Arripis georgianus, Alosa chrysochloris, Opisthonema libertate, Opisthonema oglinum, Atlantic herring (Clupea harengus), Baltic herring (Clupea harengus membras); the sardines, e.g., Ilisha species, Sardinella species, Amblygaster species, Opisthopterus equatorialis, Sardinella aurita, Pacific sardine (Sardinops sagax), Harengula clupeola, Harengula humeralis, Harengula thrissina, Harengula jaguana, Sardinella albella, Sardinella Janeiro, Sardinella fimbriata, oil sardine (Sardinella longiceps), and European pilchard (Sardina pilchardus); the hilsas, e.g., Tenuolosa species, and the anchovies, e.g., Anchoa species (A. hepsetus, A. mitchillis), Engraulis species, Thryssa species, anchoveta (Engraulis ringens), European anchovy (Engraulis encrasicolus), Engraulis eurystole, Australian anchovy (Engraulis australis), and Setipinna phasa, Coilia dussumieri. Most of these fishes have not been commercially farmed because they are generally abundant in the oceans.

In certain embodiments, the fish population comprises fishes in the superorder Ostariophysi which include the order Gonorynchiformes, order Siluriformes, and order Cypriniformes. Non-limiting examples of fishes in this group include milkfishes, catfishes, barbs, carps, danios, zebrafish, goldfishes, loaches, shiners, minnows, and rasboras. Milkfishes, such as Chanos chanos, are plankton feeders. The catfishes, such as channel catfish (Ictalurus punctatus), blue catfish (Ictalurus furcatus), catfish hybrid (Clarias macrocephalus), Ictalurus pricei, Pylodictis olivaris, Brachyplatystoma vaillantii, Pinirampus pirinampu, Pseudoplatystoma tigrinum, Zungaro zungaro, Platynematichthys notatus, Ameiurus catus, Ameiurus melas are detritivores. The carps species included are freshwater herbivores, planktivores, and detritus feeders, e.g., common carp (Cyprinus carpio), Chinese carp (Cirrhinus chinensis), black carp (Mylopharyngodon piceus), silver carp (Hypophthalmichthys molitrix), bighead carp (Aristichthys nobilis) and grass carp (Ctenopharyngodon idella). Other useful herbivores, plankton and detritus feeders are members of the Labeo genus, such as but not limited to, Labeo angra, Labeo ariza, Labeo bata, Labeo boga, Labeo boggut, Labeo porcellus, Labeo kawrus, Labeo potail, Labeo calbasu, Labeo gonius, Labeo pangusia, and Labeo caeruleus.

In a preferred embodiment, the fishes used are shiners. A variety of shiners that inhabit the Gulf of Mexico, particularly Northern Gulf of Mexico, can be used. Examples of shiners include but are not limited to, members of Luxilus, Cyprinella and Notropis genus, Alabama shiner (Cyprinella callistia), Altamaha shiner (Cyprinella xaenura), Ameca shiner (Notropis amecae), Ameca shiner (Notropis amecae), Apalachee shiner (Pteronotropis grandipinnis), Arkansas River shiner (Notropis girardi), Aztec shiner (Aztecula sallaei old), Balsas shiner (Hybopsis boucardi), Bandfin shiner (Luxilus zonistius), Bannerfin shiner (Cyprinella leedsi), Beautiful shiner (Cyprinella formosa), Bedrock shiner (Notropis rupestris), Bigeye shiner (Notropis boops), Bigmouth shiner (Hybopsis dorsalis), Blackchin shiner (Notropis heterodon), Blackmouth Shiner (Notropis melanostomus), Blacknose shiner (Can Quebec Notropis heterolepis), Blacknose shiner (Notropis heterolepis), Blackspot shiner (Notropis atrocaudalis), Blacktail shiner (Cyprinella venusta), Blacktip shiner (Lythrurus atrapiculus), Bleeding shiner (Luxilus zonatus), Blue Shiner (Cyprinella caerulea), Bluehead Shiner (Pteronotropis hubbsi), Bluenose Shiner (Pteronotropis welaka), Bluestripe Shiner (Cyprinella callitaenia), Bluntface shiner (Cyprinella camura), Bluntnose shiner (Notropis simus), Bluntnosed shiner (Selene setapinnis), Bridle shiner (Notropis bifrenatus), Broadstripe shiner (Notropis euryzonus), Burrhead shiner (Notropis asperifrons), Cahaba Shiner (Notropis cahabae), Cape Fear Shiner (Notropis mekistocholas), Cardinal shiner (Luxilus cardinalis), Carmine shiner (Notropis percobromus), Channel shiner (Notropis wickliffi), Cherryfin shiner (Lythrurus roseipinnis), Chihuahua shiner (Notropis chihuahua), Chub shiner (Notropis potteri), Coastal shiner (Notropis petersoni), Colorless Shiner (Notropis perpallidus), Comely shiner (Notropis amoenus), Common emerald shiner (Notropis atherinoides), Common shiner (Luxilus cornutus), Conchos shiner (Cyprinella panarcys), Coosa shiner (Notropis xaenocephalus), Crescent shiner (Luxilus cerasinus), Cuatro Cienegas shiner (Cyprinella xanthicara), Durango shiner (Notropis aulidion), Dusky shiner (Notropis cummingsae), Duskystripe shiner (Luxilus pilsbryi), Edwards Plateau shiner (Cyprinella lepida), Emerald shiner (Notropis atherinoides), Fieryblack shiner (Cyprinella pyrrhomelas), Flagfin shiner (Notropis signipinnis), Fluvial shiner (Notropis edwardraneyi), Ghost shiner (Notropis buchanani), Gibbous shiner (Cyprinella garmani), Golden shiner (Notemigonus crysoleucas), Golden shiner minnow (Notemigonus crysoleucas), Greenfin shiner (Cyprinella chloristia), Greenhead shiner (Notropis chlorocephalus), Highfin shiner (Notropis altipinnis), Highland shiner (Notropis micropteryx), Highscale shiner (Notropis hypsilepis), Ironcolor shiner (Notropis chalybaeus), Kiamichi shiner (Notropis ortenburgeri), Lake emerald shiner (Notropis atherinoides), Lake shiner (Notropis atherinoides), Largemouth shiner (Cyprinella bocagrande), Longnose shiner (Notropis longirostris), Mexican red shiner (Cyprinella rutila), Mimic shiner (Notropis volucellus), Mirror shiner (Notropis spectrunculus), Mountain shiner (Lythrurus lirus), Nazas shiner (Notropis nazas), New River shiner (Notropis scabriceps), Ocmulgee shiner (Cyprinella callisema), Orangefin shiner (Notropis ammophilus), Orangetail shiner (Pteronotropis merlini), Ornate shiner (Cyprinella ornata), Ouachita Mountain Shiner (Lythrurus snelsoni), Ouachita shiner (Lythrurus snelsoni), Ozark shiner (Notropis ozarcanus), Paleband shiner (Notropis albizonatus), Pallid shiner (Hybopsis amnis), Peppered shiner (Notropis perpallidus), Phantom shiner (Notropis orca), Pinewoods shiner (Lythrurus matutinus), Plateau shiner (Cyprinella lepida), Popeye shiner (Notropis ariommus), Pretty shiner (Lythrurus bellus), Proserpine shiner (Cyprinella proserpina), Pugnose shiner (Notropis anogenus), Pygmy shiner (Notropis tropicus), Rainbow shiner (Notropis chrosomus), Red River shiner (Notropis bairdi), Red shiner (Cyprinella lutrensis), Redfin shiner (Lythrurus umbratilis), Redlip shiner (Notropis chiliticus), Redside shiner (Richardsonius balteatus), Ribbon shiner (Lythrurus fumeus), Rio Grande bluntnose shiner (Notropis simus), Rio Grande shiner (Notropis jemezanus), River shiner (Notropis blennius), Rocky shiner (Notropis suttkusi), Rosefin shiner (Lythrurus ardens), Rosyface shiner (Notropis rubellus), Rough shiner (Notropis baileyi), Roughhead Shiner (Notropis semperasper), Sabine shiner (Notropis sabinae), Saffron shiner (Notropis rubricroceus), Sailfin shiner (Notropis hypselopterus), Salado shiner (Notropis saladonis), Sand shiner (Notropis stramineus), Sandbar shiner (Notropis scepticus), Satinfin shiner (Cyprinella analostana), Scarlet shiner (Lythrurus fasciolaris), Sharpnose Shiner (Notropis oxyrhynchus), Notropis atherinoides, Notropis hudsonius, Richardsonius balteatus, Pomoxis nigromaculatus, Cymatogaster aggregata, Shiner Mauritania (Selene dorsalis), Silver shiner (Notropis photogenis), Silver shiner (Richardsonius balteatus), Silver shiner (Richardsonius balteatus), Silver shiner (Notropis photogenis), Silverband shiner (Notropis shumardi), Silverside shiner (Notropis candidus), Silverstripe shiner (Notropis stilbius), Skygazer shiner (Notropis uranoscopus), Smalleye Shiner (Notropis buccula), Soto la Marina shiner (Notropis aguirrepequenoi), Spotfin shiner (Cyprinella spiloptera), Spottail shiner (Notropis hudsonius), Steelcolor shiner (Cyprinella whipplei), Striped shiner (Luxilus chrysocephalus), Swallowtail shiner (Notropis procne), Taillight shiner (Notropis maculatus), Tallapoosa shiner (Cyprinella gibbsi), Tamaulipas shiner (Notropis braytoni), Telescope shiner (Notropis telescopus), Tennessee shiner (Notropis leuciodus), Tepehuan shiner (Cyprinella alvarezdelvillari), Texas shiner (Notropis amabilis), Topeka shiner (Notropis topeka), Tricolor shiner (Cyprinella trichroistia), Turquoise Shiner (Erimonax monachus), Warpaint shiner (Luxilus coccogenis), Warrior shiner (Lythrurus alegnotus), Wedgespot shiner (Notropis greenei), Weed shiner (Notropis texanus), White shiner (Luxilus albeolus), Whitefin shiner (Cyprinella nivea), Whitemouth shiner (Notropis alborus), Whitetail shiner (Cyprinella galactura), Yazoo shiner (Notropis rafinesquei), Yellow shiner (Cymatogaster aggregata), Yellow shiner (Notropis calientis), and Yellowfin shiner (Notropis lutipinnis).

In certain embodiments, the fish population comprises fishes in the superorder Protacanthopterygii which include the order Salmoniformes and order Osmeriformes. Non-limiting examples of fishes in this group include the salmons, e.g., Oncorhynchus species, Salmo species, Arripis species, Brycon species, Eleutheronema tetradactylum, Atlantic salmon (Salmo salar), red salmon (Oncorhynchus nerka), and Coho salmon (Oncorhynchus kisutch); and the trouts, e.g., Oncorhynchus species, Salvelinus species, Cynoscion species, cutthroat trout (Oncorhynchus clarkii), and rainbow trout (Oncorhynchus mykiss); which are trophic level 3 carnivorous fish. Other non-limiting examples include the smelts and galaxiids (Galaxia speceis). Smelts are planktivores, for example, Spirinchus species, Osmerus species, Hypomesus species, Bathylagus species, Retropinna retropinna, and European smelt (Osmerus eperlanus).

In certain embodiments, the fish population comprises fishes in the superorder Acanthopterygii which include the order Mugiliformes, Pleuronectiformes, and Perciformes. Non-limiting examples of this group are the mullets, e.g., striped grey mullet (Mugil cephalus), which include plankton feeders, detritus feeders and benthic algae feeders; flatfishes which are carnivorous; the anabantids; the centrarchids (e.g., bass and sunfish); the cichlids, the gobies, the gouramis, mackerels, perches, scats, whiting, snappers, groupers, barramundi, drums wrasses, and tilapias (Oreochromis sp.). Examples of tilapias include but are not limited to nile tilapia (Oreochromis niloticus), red tilapia (O. mossambicus x O. urolepis hornorum), mango tilapia (Sarotherodon galilaeus).

In certain embodiments, certain fish provided herein have significantly higher lipid content, for example, greater than 10%, 15%, 20%, 25%, 30%, 35%, 40%, 45%, 50%, 55%, or 60%, as a result of the aquaculture methods described herein. In certain embodiments, certain fish provided herein have significantly higher EPA and/or DHA concentrations, for example, greater than 25%, 30%, 35%, 40%, 45%, 50%, 55%, or 60%, as a result of the aquaculture methods described herein. In certain embodiments, certain fish provided herein have significantly higher EPA and/or DHA yields, for example, greater than 25%, 30%, 35%, 40%, 45%, 50%, 55%, 60%, 65%, 70%, 75%, 80%, 85%, 90%, 95%, 96%, 97%, 98%, or 99%, as a result of the aquaculture methods described herein. Without being bound by any particular theory, the improvements may be achieved by the reduced metabolism of captive fish and the higher EPA and/or DHA in their diets. As a comparison, the yield of fish oil from wild menhaden is typically 10% of the wet fish weight (United States Securities and Exchange Commission Form 10-K Report, Omega Protein Corporation, 2008), and the oil contain 23% EPA and/or DHA (“Product Specifications for Menhaden Fish Oil,” Omega Protein Corp., May 15, 2008).

Algae are used as feed for larvae of certain shellfish that are used as human food, e.g., Mercenaria species (clams), Crassostrea species (oysters), Ostrea species, Pinctada species, Mactra species, Haliotis species (abalone), Pteria species, Patinopecten species (scallops). Invertebrate shellfish, bivalves, mollusks may reside in or be present within the enclosures of the embodiments, but they are not contemplated as a part of the present embodiments.

The following non-limiting examples of fish species can be used to harvest algae in or near the Gulf of Mexico: Brevoortia species such as B. patronus and B. tyrannus, species within Luxilus, Cyprinella and Notropis genus, Hyporhamphus unifasciatus, Sardinella aurita, Adinia xenica, Diplodus holbrooki, Dorosoma petenense, Lagodon rhombodides, Microgobius gulosus, Mugil species such as Mugil cephalus, Mugil cephalus, Mugil curema, Sphoeroides species such as Sphoeroides maculatus, Sphoeroides nephelus, Sphoeroides parvus, Sphoeroides spengleri, Aluterus schoepfi, Anguilla rostrata, Arius felis, Bairdella chrysoura, Bairdeiella chrysoura, Chasmodies species such as Chasmodes saburrae and Chasmodies saburrae, Diplodus holbrooki, Heterandria formosa, Hybopsis winchelli, Ictalurus species such as Ictalurus serracantus and Ictalurus punctatus, Leiostomus xanthurus, Micropogonias undulatus, Monacanthus ciliatus, Notropis texanus, Opisthonema oglinum, Orthopristis chrysoptera, Stephanolepis hispidus, Syndous foetens, Syngnathus species such as Syngnathus scovelli, Trinectes maculatus, Archosargus probatocephalus, Carpiodes species such as C. cyprinus and C. velifer, Dorosoma cepedianum, Erimyzon species such as Erimyzon oblongus, Erimyzon sucetta, and Erimyzon tenuis, Floridichthys carpio, Microgobius gulosus, Monacanthus cilatus, Moxostoma poecilurum, and Orthopristis chrysophtera.

Transgenic fish and genetically improved fish can also be used in the harvesting methods provided herein. The term “genetically improved fish” refers herein to a fish that is genetically predisposed to having a higher growth rate and/or a lipid content that is higher than a wild type fish, when they are cultured under the same conditions. Such fishes can be obtained by traditional breeding techniques or by transgenic technology. Over-expression or ectopic expression of a piscine growth hormone transgene in a variety of fishes resulted in enhanced growth rate. For example, the growth hormone genes of Chinook salmon, Sockeye salmon, tilapia, Atlantic salmon, grass carp, and mud loach have been used in creating transgenic fishes (Zbikowska, Transgenic Research, 12:379-389, 2003; Guan et al., Aquaculture, 284:217-223, 2008). Transgenic carp or transgenic tilapia comprising an ectopically-expressed piscine growth hormone transgene are particularly useful in the harvesting methods provided herein.

5.3 Methods and Systems

Described below are useful methods and systems of the present embodiments for producing EPA and/or DHA from algae. In various embodiments, certain methods provided herein comprise harvesting algae that produce EPA and/or DHA by feeding the algae to a population of fishes, extracting lipids from the fishes; and separating the EPA and/or DHA from the lipids. As used herein the term “system” or “enclosed-container system” refers generally to one or more installations, enclosures, containers, tanks, vessels, or apparatus for practicing the methods provided herein. As used herein, the term “open-pond system” refers to any system of one or more open ponds or raceways. In certain embodiments, the systems provided herein comprise water containing-enclosures that provide a multi-tropic aquatic environment that supports the growth of algae and/or planktivorous organisms, such as fishes, and can emulate various aspects of an ecological system. In certain embodiments, the systems further comprise means for feeding algae to a population of fish thereby harvesting the algae, means for extracting lipids from the fish; and means for converting the lipids to EPA and/or DHA, and optionally means for culturing algae. In certain embodiments, the systems can comprise, independently and optionally, means for monitoring and/or controlling the aquatic environment in the enclosures, means for maintaining algal stock cultures, means for maintaining fish stocks, means for concentrating algae, means for storing algal biomass, means for storing fish biomass, means for conveying algae to fish, means for conveying fish to processing, means for separating lipids from fish biomass, and means for separating and purifying EPA and/or DHA from the other lipids.

The term “fish enclosure” refers to a water-containing enclosure in which cultured algae are harvested by fish. The term “growth enclosure” refers to a water-containing enclosure in which the algae are grown and/or stored in water. Most of the algal growth takes place in the growth enclosure which is designed and equipped to optimize algal growth. Depending on the environment and economics of the operation, the methods and systems for harvesting algae can be integrated with the culturing of algae. In one embodiment, the algae and fishes are cultured in the same enclosure wherein the fishes and algae commingle in the same body of water, and the fishes in the enclosure feed on the algae. The algae are cultured in the enclosure so the enclosure preferably has a surface area and depth that allow exposure of the algae to light. In this embodiment, the growth enclosure and the fish enclosure are effectively the same enclosure. In a particular embodiment, the fishes and the algae reside in the same enclosure but the fishes are confined or caged in a zone within the enclosure. The fishes are gathered periodically or continuously from the enclosure to produce EPA and/or DHA.

In another embodiment, the algae and the fishes are cultured separately for at least a period of time before the algae are fed to the fishes. Algae are cultured in a growth enclosure and are made available in batches or continuously to fishes, which are separately kept in a fish enclosure. The algae in its growth enclosure can be but are not limited to a monoculture, a mixed algal culture, a mixed algal and fish culture, or a photobioreactor. The algae may share the same body of water in a system with the fish. An aquatic composition comprising algae can be introduced into a fish enclosure in which harvesting fishes reside, and later returned to the growth enclosure that contains the bulk of the algae. Alternatively, the algae and the fish do not use the same body of water until the algae are fed to the fishes. Accordingly, in certain embodiments, the methods can comprise the step of culturing the algae, culturing the fish, or culturing both, separately or together, in an enclosure.

The enclosures of the present embodiments contain an aquatic composition comprising algae and/or fishes, and are means for confining the algae and/or fishes in an aquatic environment at a location on land, in a body of water, or at sea. The enclosures can be but are not limited to plastic bags, carboys, raceways, channels, tanks, cages, net-pens, ponds, and artificial streams. The enclosure can be of any regular or irregular shape, including but not limited to rectangular tanks, cages or ponds, or circular tanks, cages or ponds. A cage can be submerged, submersible or floating in a body of water, such as a lake, a bay, an estuary, or the ocean. A pond can be unlined or lined with any water-permeable materials, including but not limited to, cement, polyethylene sheets, or polyvinylchloride sheets. Example of ponds includes but are not limited to earthen pond, lined pond, barrage pond, contour pond, and paddy pond. Erecting barriers that separate a water-containing area from a natural body of water can also form a pond. Segregating a body of water by embankments, partitions and/or nets can form an enclosure. Cages, net-pens and such like are used to confine the movement of the fish in an enclosure, or used as an enclosure in a body of water. The enclosures, such as ponds, can be organized in tracks on land, and cages can be organized in clusters in lakes or at sea so that they can share a host of operational and maintenance equipment. Fishes of different trophic types, species, sizes, or ages, can be cultured separately in enclosures, cages, and net-pens.

In addition to algae and fishes, in certain embodiments, the enclosures provided herein may comprise one or more additional aquatic organisms, such as but not limited to bacteria; plankton including zooplankton, such as but not limited to larval stages of fishes (i.e., ichthyoplankton), tunicates, cladocera and copepoda; crustaceans, insects, worms, nematodes, mollusks and larval forms of the foregoing organisms; and aquatic plants. This type of culture system emulates certain aspects of an ecological system and is referred to as a multi-trophic system. The bacteria, plants, and animals constitute various trophic levels, and lend stability to an algal culture that is maintained in the open. These organisms can be introduced into the system or they may be present in the environment in which the culture system is established. However, zooplankton graze on microalgae and are generally undesirable if present in excess in an enclosure of the present embodiments. In certain embodiments, they can be removed from the water by sand filtration or by keeping zooplanktivorous fishes in the enclosure. The numbers and species of plankton, including zooplanktons, can be assessed by counting under a microscope using, for example, a Sedgwick-Rafter cell.

The growth enclosure(s) and/or fish enclosure(s) of the systems of the present embodiments can each be closed or open, or a combination of open and closed enclosures. The enclosures can be completely exposed, covered, reversibly covered, or partly covered. The communication or material flow between a closed enclosure and its immediate aquatic and/or atmospheric environment is highly controlled relative to an open enclosure. Systems comprising open enclosures can be multi-trophic systems, with or without means for environmental controls. The size of an open enclosure of the embodiments can range, for example, from about 0.05 hectare(ha) to 20 ha, from about 0.25 to 10 ha, and preferably from about 1 to 5 ha. Systems comprising open enclosures that are situated on land can comprise one or more growth enclosure(s)and/or fish enclosure(s), which can be independently, ponds and/or raceways. The depth of such systems can range, for example, from about 0.3 m to 4 m, from about 0.8 m to 3 m, and from about 1 to 2 m. Raceways can be operated at shallow depths of 15 cm to 1 m. Typical dimensions for raceways are about 30:3:1 (length:width:depth) with slanted or vertical sidewalls. The systems can comprise a mix of different physical types of enclosures. The enclosures of the embodiments can be set up according to knowledge known in the art, see, for example, Chapters 13 and 14 in Aquaculture Engineering, Odd-Ivar Lekang, 2007, Blackwell Publishing Ltd., respectively, for description of closed culturing systems and open culturing systems.

Most natural land-based water sources, such as but not limited to rivers, lakes, springs and aquifers, and municipal water supply can be used as a source of water for used in the systems provided herein. Seawater from the ocean or coastal waters, artificial seawater, brackish water from coastal, estuarine regions, or impaired underground aquifers can also be a source of water. Irrigation water, eutrophic river water, eutrophic estuarine water, eutrophic coastal water, agricultural wastewater, industrial wastewater, or municipal wastewater can also be used in the systems provided herein. Optionally, one or more effluents of the system can be recycled within the system. The systems of the embodiments provided herein optionally comprise means for connecting the enclosures to each other, to other parts of the system and to water sources and points of disposal. The connections permit the operators to move and exchange water between parts of the system either continuously or intermittent, as needed. The connecting means, temporary or permanent, facilitates fluid flow, and can include but is not limited to a network of channels, hoses, conduits, viaducts, and pipes. The systems further comprise means for regulating the rate, direction, or both the rate and direction, of fluid flow throughout the network, such as flow between the enclosures and between the enclosures and other parts of the system. The flow regulating means can include but is not limited to pumps, valves, manifolds, and gates. Optionally, effluents from one or more enclosures are recycled generally within the system, or selectively to certain parts of the system.

The systems of the embodiments provided herein also provide means to monitor and/or control the environment of the enclosures, which includes but is not limited to the means for monitoring and/or adjusting, independently or otherwise, the pH, salinity, dissolved oxygen, alkalinity, nutrient concentrations, water homogeneity, temperature, turbidity, and other conditions of the water. The fish enclosures of the embodiments can operate within the following non-limiting, exemplary water quality limits: dissolved oxygen at greater than 5 mg/L, pH 6-10 and preferably pH from 6.5-8.2 for cold water fishes and pH7.5 to 9.0 for warm water fishes; alkalinity at 10-400 mg/L CaCO₃; salinity at 0.1-3.0 g/L for stenohaline fishes, 0.1 to 35 g/L for euryhaline, and 28-35 g/L for marine fishes; less than 0.5 mg NH₃/L; less than 0.2 mg nitrite/L; and less than 10 mg/L CO₂ Equipment commonly employed in the aquaculture industry, such as thermometers, thermostats, pH meters, conductivity meters, dissolved oxygen meters, and automated controllers can be used for monitoring and controlling the aquatic environments of the system. For example, the pH of the water is preferably kept within the ranges of from about pH 6 to pH 9, and more preferably from about 8.2 to about 8.7. The salinity of seawater ranges preferably from about 12 to about 40 g/L and more preferably from 20 to 24 g/L. The temperature for seawater-based culture ranges preferably from about 16° C. to about 27° C. or from about 18° C. to about 24° C.

Generally, oxygen consumption by fish increases shortly after feeding, and water temperature regulates the rate of metabolism. The oxygen transport rate from water to fish is directly dependent on the partial oxygen pressure differences between fish blood (e.g., 50-110 mmHg) and the dissolved oxygen concentration in local water (e.g., 154-158 mmHg at sea level). During the day, the algae will provide oxygen and the fish will provide the carbon dioxide. At night, both algae and fish will respire and may require active oxygenation. The systems provided herein can comprise means for delivering a gas or a liquid comprising a dissolved gas to the water in the systems, which include but are not limited to hoses, pipes, pumps, valves, and manifolds. Bubbles in the culture media can be formed by injecting gas, such as air, using a jet nozzle, sparger or diffuser, or by injecting water with bubbles using a venturi injector. Various techniques and means for oxygenation of water known in the art can be applied in the methods provided herein, see, for example, Chapter 8 in Aquaculture Engineering, Odd-Ivar Lekang, 2007, Blackwell Publishing Ltd. The addition of carbon dioxide promotes photosynthesis, and helps to maintain the pH of the culture below pH 9. Sources of carbon dioxide include, but is not limited to, synthetic fuel plants, gasification power plants, oil recovery plants, ammonia plants, ethanol plants, oil refinery plants, anaerobic digestion units, cement plants, and fossil steam plants. Carbon dioxide, either dissolved or as bubbles, at a concentration from about 0.05% to 1%, and up to 5% volume of air, can be introduced into the enclosures. Other instruments and technology for monitoring aquatic environments known in the art can be applied in the methods and systems provided herein, see, for example, in Chapter 19 of Aquaculture Engineering, Odd-Ivar Lekang, 2007, Blackwell Publishing Ltd.

Depending on the source of water, it may be necessary to provide additional nutrients to sustain algal growth in the enclosures of the present embodiments. The growth enclosures can be fertilized regularly according to conventional fishery practices. Nutrients can be provided in the form of fertilizers, including inorganic fertilizers, such as but not limited to, ammonium sulfate, urea, calcium super phosphate, sodium metasilicate, sodium orthosilicate, sodium pyrosilicate, and silicic acid; and organic fertilizers, such as but not limited to, manure and agricultural waste.

The methods of the present embodiments comprise a step of harvesting algae by feeding the algae to fish. The feeding of algae to fish encompasses any methods by which the algae and fishes of the present embodiments are brought into proximity of each other such that the fishes can ingest the algae. Preferably, the systems are designed to make the algae accessible to the fishes in an energy-efficient and controlled manner. The algae in an algal composition can be added to, pumped into, or allowed to flow into an enclosure in which the fishes are held. An algal composition can be made available to the fishes in batches or on a continuous basis. The algae can be distributed throughout the fish enclosure by any means, such as but not limited to agitation or aeration of the enclosure. The algae can also be dispensed at multiple locations in the fish enclosure. The algae can be distributed by water current in the enclosure in which the fishes swim through.

While the fishes are feeding on the algae, they may be swimming freely in the enclosure or they may be confined in one or more zones within the enclosure. The size and number of the zones in the fish enclosure may be controlled to adjust the density of fish per unit volume (e.g., in a chamber) or unit area (e.g., in a shallow enclosure). The zones may be established by membranes, nets, fixed cages, floating cages, partitions, or other means known in the art. The fish enclosure or zones therein provides several advantages. First, the enclosure or zone can be covered by netting to minimize predation by birds. Second, the enclosure or zone also allows simple harvesting by seining. Third, the enclosure or zone can limit the overconsumption of algae by the fish. However, still water is generally not preferred as it allows stratification and accumulation of waste products. In one embodiment, the fish enclosure is not zoned. In another embodiment, the algae flow past the fishes within the fish enclosure or zones. Preferably, the fishes within the enclosures or zones remain relatively stationary. In yet another embodiment, the fishes are allowed access to the algae, for example, by allowing the fishes to swim from one gated enclosure to the algae in another enclosure, or allowing the fishes to swim to another zone within the enclosure that was not previously accessible. In yet another embodiment, the total number of fishes or the number of a species of fishes in an enclosure or a zone is increased or decreased. In certain embodiments, the system is designed to minimize the energy that would be expended by the fishes to acquire the algae, and to reduce physiological stress, such as overcrowding, low oxygen and waste accumulation. The systems provided herein comprise means for controlling the movement of fishes in the system, means for adding fishes to or removing fishes from the system, such as but not limited to gates, channels, and portals, and means for removing dissolved and solid wastes (e.g., pumps and sinks), means for adding, removing, or relocating cages containing fishes. Conventional fish hatcheries and farming techniques known in the art can be applied to implement the systems and methods provided herein, see for example, Chapters 10, 13, 15 in Aquaculture Engineering, Odd-Ivar Lekang, 2007, Blackwell Publishing Ltd.

It should be understood that the enclosure in which the fishes are kept prior to feeding likely contains some algae at a background level. When the algae is added, pumped, or delivered to the water in which the fishes are kept, the total amount of algae in the fish enclosure—the concentration of algae will rise above the background level initially. If the algae is not provided continuously, the amount of algae in the fish enclosure may decrease following feeding by the fishes over a period of time. This situation also arises when the fishes are allowed access to the algae by swimming to a fish enclosure that comprises the algae.

The algae can be delivered to the fishes directly from an algal culture or it can be concentrated prior to being provided to the fishes. The concentration of an algal composition can range from about 0.01 g/L, about 0.1 g/L, about 0.2 g/L, about 0.5 g/L to about 1.0 g/L. It should be understood that the concentration step does not require, nor does it exclude, that the algae be dried, dewatered, or reduced to a paste or any semi-solid state. The concentration step can be performed serially by one or more different techniques to obtain a concentrated algal composition. The concentration step serves the purpose of reducing the energy cost of transporting the algae to the fishes and to reduce the volume of water that is transferred into the fish enclosure. A concentrated algal composition may be stored for a period of time, or fed to the fishes immediately. It is contemplated that different batches of algae can be combined to form one or more algal compositions before the algae are being harvested in the fish enclosure. The algal composition can comprise different groups of algae in defined or undefined proportions. An algal composition can be designed to enhance the growth of the fishes and/or the accumulation of lipids in the fishes. In various embodiments, the algae are concentrated so that the number of algal cells per unit volume increases by two, five, 10, 20, 25, 30, 40, 50, 75, 100-fold, or more. For example, after a concentration step, the concentration of algae in an algal composition can range from at least about 0.2 g/L, about 0.5 g/L, about 1.0 g/L, about 2.0 g/L, about 5 g/L to about 10 g/L. An algal composition of the present embodiments can be a concentrated algal culture or composition that comprises about 110%, 125%, 150%, 175%, 200% (or 2 times), 250%, 500% (or 5 times), 750%, 1000% (10 times) or 2000% (20 times) the amount of algae in the original culture or in a preceding algal composition. The algae can also be dried to remove most of the moisture (water <1%). The resulting concentrated algae composition can be a solid, a semi-solid (e.g., paste), or a liquid (e.g., a suspension), and it can be stored or used to make EPA and/or DHA immediately. The concentrated algal composition can be held in one or more separate enclosures. Any techniques and means known in the art for concentrating the algae can be applied, including but not limited to centrifugation, filtration, sedimentation, flocculation, and foam fractionation. See, for example, Chapter 10 in Handbook of Microalgal Culture, edited by Amos Richmond, 2004, Blackwell Science, for description of downstream processing techniques.

The fishes of the embodiments provided herein are selected to maintain the feed conversion ratio (FCR) within a range that can optimize the biomass production as well as accumulate lipids, especially EPA and/or DHA. The FCR is calculated from the kilograms of feed that are used to produce one kilogram of whole fish, and reflects how efficiently the feed is converted into fish biomass. The particular value of FCR is based, in part, on the metabolism of the particular species of fish, the digestibility of the food, its nutritional characteristics, and the quantity of food. Overfeeding or underfeeding a fish can vary the FCR, while feeding a fish to satiation can reduce the FCR because satiated fish are not stressed, and produce dense, high quality flesh. Thus, controlling the concentration and species composition of algae on which the fishes feed can be useful for optimizing the FCR, such as by reducing the FCR in a system. The FCR can also depend on the particular food source, for example, some fish species are particularly well adapted to using oils and fats as their prime energy source. Thus, selecting algae species with a high oil/fat content can reduce the FCR for a species of fish. In some embodiments, the species of fish has an FCR of less than about 3, less than about 2, less than about 1.5, less than about 1.0, less than about 0.8, or less than 0.6.

A feeding regimen can be established to encourage the feeding of the fishes on the algae to a predetermined ration level or to satiation, in order to accelerate the growth rate, and to maximize gain in fish biomass. For example, an excess of algae is made available to the fishes up to or above the limiting maximum stomach volume of the fishes. The feeding process, water temperature in the fish enclosure, the growth of fishes in size and/or in biomass, and the accumulation of lipids, can be monitored, quantified and tabulated by methods well known in the art. Energy requirements of fish are calculated from maintenance requirements (fasted animals), growth rate, water temperature, and losses during food utilization (Cho, Aquaculture, 100:107-123, 1992). The collected data, for example, in the form of a feeding table, can be used to fine-tune various parameters of the system to maximize biomass yield. The systems of the present embodiments provide means for feeding a controlled amount of algae to the fishes. The systems of the embodiments can provide a feeding subsystem to control the feeding of algae to the fishes. Many feeding mechanisms are known in the art, see e.g., Chapter 16, Aquaculture Engineering, Odd-Ivar Lekang, 2007, Blackwell Publishing Ltd.

The density of algae in the fish enclosure can be monitored and adjusted to promote feeding at a predetermined rate or to satiation, such as by maintaining the density at a constant level that is at least about 50%, about two times, about three times, about five times, about 10 times, about 20 times, or about 50 times the average amount of algae normally present in a natural aquatic environment, such as a local aquatic environment in which the endemic species coexist. For example, the algae can be present at a concentration of greater than about 10, 25, 50, 75, 100, 250, 500, 750, 1000 mg/L, or about 10 to about 500 mg/L, about 50 to about 200 mg/L, or about 200 to 1000 mg/L, based on ash-free dry weight. In embodiments where the fishes are fed in a batchwise manner, the algae may be provided once a day, twice a day, once a week, twice a week, or three times a week, or whenever the density of algae in the fish enclosure falls below a predetermined level. The algae in the fish enclosure are the major source of food that provide energy and support growth of the fishes, although natural bodies of water will contain phytoplanktivorous organisms, such as zooplankton, which also serve as food for the fish. In essence, the zooplankton serve as an intermediary algae harvester. Vitamins, such as thiamin, riboflavin, pyroxidine, folic acid, panthothenic acid, biotin, inositol, choline, niacin, vitamin B12, vitamin C, vitamin A, vitamin D, vitamin E, vitamin K; and minerals, such as but not limited to calcium, phosphorous, magnesium, iron, copper, zinc, manganese, iodine and selenium, required for optimal fish growth which may not be sufficiently provided by the algae, and other aquaculture additives, such as antibiotics, may be provided separately. Preferably, while the fishes are consuming the algae, the fishes in the enclosure are provided with a minimum, if any, of other aquaculture feedstuff (e.g., agricultural feedstuff, silage, pelleted commercial intensive feeds) to provide energy and sustain growth. In certain embodiments, the fishes are fed exclusively cultured algae, optionally presented in the form of a concentrated algal composition. The systems of the invention also comprise means for providing supplemental aquaculture feed and aquaculture additives to the fishes, such as various types of automated feeders, including demand feeders, adaptive feedback feeders, and fixed ration feeders. The feeders can also be adapted to supply the fishes with algae of the present embodiments.

Depending on the site and the type of fish used, for a system comprising open enclosures, the fish can be introduced at various density from about 50 to 100, about 100 to 300, about 300 to 600, about 600 to 900, about 900 to 1200, and about 1200 to 1500 individuals per m². The enclosures of the embodiments can be characterized by their loading density and carrying capacity. The loading density of a fish enclosure is the total fish biomass housed within the enclosure. The carrying capacity is the fish biomass in the enclosure without compromising water quality, fish nutrition, or fish health. Carrying capacity is a function of water flow, enclosure volume, exchange rate, rearing temperature, dissolved oxygen, metabolic wastes (e.g., ammonia), which can be adjusted by techniques known in the art. Loading density and carrying capacity are measured either by a density index (in units of fish weight per volume/space, e.g., lb/cubic feet, kg/ha) or by a water flow index governed by oxygen consumption (in units of fish weight per volume per minute, kg/L/min). For example, the loading density ranges from about 0.5 to 1 pound of fish per 2 gallons of water with saturated oxygen levels.

As the fishes feed on algae and grow over time, the carrying capacity of an enclosure may not be adequate. It is contemplated that the fishes may be transferred from a first enclosure to a second enclosure with a larger carrying capacity to reduce stress and thus allow the fishes to grow rapidly. The loading density of the second enclosure is initially lower than that of the first enclosure. The algae consumption by the population of fish cannot exceed the algae production rate or else algae population will crash. As the population of fish grow, their algae consumption will also increase and therefore the number of fish needs to be removed from the system by either harvesting or transferring to a different enclosure. Depending on the age of the fishes, they may be transferred successively to various enclosures of the system with different, possibly larger, carrying capacities. The transfer can be effected by allowing the fishes to swim from one enclosure to another enclosure or manual capture (e.g., netting) and movement. Alternatively, the growing fish population may be divided periodically among several enclosures. The residence time in each water enclosure depends on the growth rate and the carrying capacity of the enclosure. If the system is designed such that various aspects of water quality can be adjusted, the fishes may remain in an enclosure while the parameters within the enclosure are changed to accommodate the needs of growing fishes. In one embodiment, the enclosure is maintained at carrying capacity until just before the fishes is ready for processing when the enclosure is switched to operating towards maximizing loading density.

Depending on the growth rate and life cycle of the fishes, they can be gathered at any time after they have fed on the algae and gained sufficient biomass for fish oil and fishmeal processing, or to mitigate against overgrazing. It is contemplated that fish fry, juveniles, fingerlings, and/or adult fish, can be used initially to stock the fish enclosure. As the fish fry, fingerlings or juveniles become adults that have grown to reach or exceed a desired biomass, they are gathered from the enclosure and optionally, kept in a separate holding enclosure. In one embodiment, the fishes are gathered when a certain percentage of fishes in the population reach maturity, or when the biomass of a percentage of the fishes reaches a predetermined level referred to herein as a biomass set point. The percentage of fish in the population that reaches or exceeds the set point can be at least about 20%, at least about 30%, at least about 40%, at least about 50%, at least about 60%, at least about 70%, at least about 80%, at least about 90% or at least about 95%. Various sampling methods known in the art can be used to assess the percentage for a population of fishes.

A fish biomass set point, measurable in teens of the gain of biomass over a period of time, is used to determine the time when the fishes are gathered or captured for processing.

In one embodiment, the set point can be the average or median biomass of an adult fish of one of the major fish species in the population. The set point can be the weight, length, body depth, or fat content of the fish at a certain age ranging from 2 weeks old to 3 years old or more, such as but not limited to, 2 weeks, 4 weeks, 8 weeks, 3 months, 6 months, 9 months, 12 months, 15 months, 18 months, 21 months, or 24 months. For example, the set point can be the 2-week weight, 2-week length, 2-week body depth, 2-week fat content, 4-week weight, 4-week length, 4-week body depth, 4-week fat content, 8-week weight, 8-week length, 8-week body depth, 8-week fat content, 3-month weight, 3-month length, 3-month body depth, 3-month fat content, 6-month weight, 6-month length, 6-month body depth, 6-month fat content, 9-month weight, 9-month length, 9-month body depth, 9-month fat content, 12-month weight, 12-month length, 12-month body depth, 12-month fat content, 15-month weight, 15-month length, 15-month body depth, 15-month fat content, 18-month weight, 18-month length, 18-month body depth, 18-month fat content, 21-month weight, 21-month length, 21-month body depth, 21-month fat content, 24-month weight, 24-month length, 24-month body depth, or 24-month fat content of one of the major species of fish in the enclosure. In another embodiment, the set point can be the biomass of one of the major species of fish when the growth rate of the species reaches a plateau under the culture conditions in the fish enclosure. The set point can also be based on the biomass of separate parts of a fish, e.g. fish fillet, fish viscera, head, liver, guts, testes, and ovary. The fillet weight and viscera weight of a fish can be measured to monitor growth. The lipid content of the fillet and viscera of the fishes can be determined by methods known in the art, and are typically within the range of about 10%-20% (fillet) and 10% to 40% (viscera) by weight.

In another embodiment, provided herein are systems and methods that are based on co-culturing both the algae and the fishes in an enclosure while the fishes feed continuously on the algae and the aquatic conditions in the enclosure are optimized so that the productivity of algal biomass is maintained at a maximum level over a period of time. The productivity of such systems is governed in part by the density of algae in the enclosure which is affected both by the growth rate of the algae and the feeding rate of the fishes. As the fishes in an enclosure grow to maturity, the feeding rate increases and can significantly reduce the density of the algae, and affect the productivity of the system over a period of time. To maintain the productivity of the system (measurable by e.g., algal biomass gained per unit volume per unit time) at a desired level, it is preferable to maintain the density of the algae (measurable by e.g., algal biomass per unit area or unit volume) at a constant level or within a defined range, i.e., a set point based on algal biomass. An algal biomass set point can be the concentration of algae in an enclosure or a zone thereof, which can range from 1 to 1000 mg/L, including but not limited to 2, 5, 10, 20, 30, 40, 50, 100, 150, 200, 250, 300, 350, 400, 450, 500, 550, 600, 650, 700, 750, 800, 850, 900, 950, or 1000 mg/L.

This can be achieved by controlling the feeding rate of the fishes or the number or size of fishes in the enclosure. In such systems, the fishes are preferably confined to a zone or in cages, such that the total number of fishes or the number of a species of fishes can be monitored and regulated. In a specific embodiment, the productivity or the density of algae in an enclosure determines the total number of fishes, the size distribution of one or more species of fishes, the age distribution of one or more species of fishes, or the time when a plurality of the fishes is gathered and removed from the system. The age range of the fishes can be from 2 weeks old to 3 years old or more, such as but not limited to, 2 weeks, 4 weeks, 8 weeks, 3 months, 6 months, 9 months, 12 months, 15 months, 18 months, 21 months, or 24 months. The size range of the fishes can be measured in terms of length or body depth as described above for fish biomass set point. In another embodiment, regulating the flow rate of algae to the fishes in an enclosure or a zone thereof, or in cages controls the feeding rate. The flow rate of algae can be regulated by changing the degree of mixing in an enclosure or in the vicinity of a zone or a cage. Accordingly, the methods provided herein comprise increasing or decreasing the total number of fishes, the number of one or more species of fishes, the number of fishes of a defined size range, or the number of fishes of a defined age range, in an enclosure, a zone thereof, or a cage. In a specific embodiment, one or more cages of fish can be added to or removed from an enclosure.

The total residence time of a fish population in one or more fish enclosures of the system wherein the fishes are fed with the algae may range from about 30 to 90 days, about 12 to 24 weeks, or about 6 to 24 months. The fishes can be gathered or harvested by any methods or means known in the art. In some embodiments, a fish gathering or capturing means is configured to separate fish based on a selected physical characteristic, such as density, weight, length, or size. The harvesting systems of the embodiments comprise means to gather or capture fish, which can be mechanical, pneumatic, hydraulic, electrical, or a combination of mechanisms. In one embodiment, the fish gathering device is a net that is either automatically or manually drawn through the water in order to gather or capture the fishes. The net, with fishes therein, can then be withdrawn from the pond. Alternately, a fish gathering device can comprise traps, or circuits for applying DC electrical pulses to the water. For example, see Chapters 17 and 19 in Aquaculture Engineering, Odd-Ivar Lekang, 2007, Blackwell Publishing Ltd., for description of techniques and means for moving and grading fish.

Any fish processing technologies and means known in the art can be applied to obtain lipids and fishmeal from the fishes. In one embodiment, the entire body of a fish is used in making lipids and fishmeal. The entire fish is processed to extract lipids without separating the fish fillet from other parts of the fish, which are regarded as fish waste in the seafood industry. In another embodiment, only certain part(s) of the fish are used, e.g., non-fillet parts of a fish, fish viscera, head, liver, guts, testes, and/or ovary. Prior to being processed, the fishes of the present embodiments are not treated to prevent or remove off-flavor taste of the flesh. The treatment may include culturing the fishes for a period from one day up to two weeks in an enclosure that has a lower algae and/or bacteria count than the fish enclosure.

Described below is an example of a method for processing the fishes of the present embodiments. The processing step involves heating the fishes to greater than about 70° C., 80° C., 90° C. or 100° C., typically by a steam cooker, which coagulates the protein, ruptures the fat deposits and liberates lipids and oil and physico-chemically bound water, and; grinding, pureeing and/or pressing the fish by a continuous press with rotating helical screws. The fishes can be subjected to gentle pressure cooking and pressing which use significantly less energy than that is required to obtain lipids from algae. The coagulate may alternatively be centrifuged. This step removes a large fraction of the liquids (press liquor) from the mass, which comprises an oily phase and an aqueous fraction (stickwater). The separation of press liquor can be carried out by centrifugation after the liquor has been heated to 90° C. to 95° C. Separation of stickwater from oil can be carried out in vertical disc centrifuges. The lipids in the oily phase (fish oil) may be polished by treating with hot water which extracts impurities from the lipids. To obtain fish meal, the separated water is evaporated to form a concentrate (fish solubles) which is combined with the solid residues, and then dried to solid form (presscake). The dried material may be grinded to a desired particle size. The fish meal typically comprises mostly proteins (up to 70%), ash, salt, carbohydrates, and oil (about 5-10%). The fish meal can be used as animal feed and/or as an alternative energy feedstock.

In certain embodiments, fish meal can be produced from fish bodies and processing residue thereof by optionally pretreating, for example, cutting, crushing or grinding the raw material; boiling the treated material; pressing the same to thereby separate liquid matters containing a fish oil; drying the residual solid matters optionally together with fish-solubles, which will be described hereinafter; grinding the material, if required, to thereby give a fish meal; while separating the fish oil from the liquid matters; and concentrating the residual liquid matters to thereby produce fish-solubles.

In certain embodiments, fish meal can be produced from treating fish bodies with a protease acting at a relatively low temperature. In certain embodiments, proteases that can be used include proteinases such as acrosin, urokinase, uropepsin, elastase, enteropeptidase, cathepsin, kallikrein, kininase 2, chymotrypsin, chymopapain, collagenase, streptokinase, subtilisin, thermolysin, trypsin, thrombin, papain, pancreatopeptidase and rennin; peptidases such as aminopeptidases, for example, arginine aminopeptidase, oxytocinase and leucine aminopeptidase; angiotensinase, angiotensin converting enzyme, insulinase, carboxypeptidase, for example, arginine carboxypeptidase, kininase 1 and thyroid peptidase, dipeptidases, for example, carnosinase and prolinase and pronases; as well as other proteases, denatured products thereof and compositions thereof

In certain embodiments, the extracted fish oil can contain EPA and/or DHA ranging from 1 to 50%, depending on the fish species, age, location, and a host of ecological and environmental factors. If higher EPA and/or DHA concentrations are desired, several established methods could be employed, including chromatography, fractional or molecular distillation, enzymatic splitting, low-temperature crystallization, supercritical fluid extraction, or urea complexation. These methods can further concentrate the EPA and/or DHA to nearly pure EPA and/or DHA.

In certain embodiments, EPA- and/or DHA-containing lipids may be separated and concentrated by short-path distillation, or molecular distillation. See e.g., Albers, M. and Graverbolt, J. P., “Short-path distillation in the fish oil industry” UIC GmbH, 2006. The lipids are first transesterified, either acid- or base-catalyzed, with ethanol to produce a mixture of fatty acid ethyl esters (FAEE). The FAEE are then fractionated in the short-path distillation to remove the short chain FAEE, C-14 to C-18. The concentrate of FAEE from C-20 to C-22 is where the EPA and/or DHA can be found. A second distillation of the concentrate can result in a final Omega-3 content of up to 70%. The concentration of the EPA and/or DHA in the final product will depend on the initial lipid profile of the fish oil. The FAEE can be used as a consumer product at this stage (fish oil capsules). In some countries, the FAEE are required to be reconverted to triglycerides through a glycerolysis reaction before they can be sold as a consumer product. In order to obtain pure EPA and/or DHA, an additional purification step is required using chromatography, enzymatic transesterification, ammonia complexation, or supercritical fluid extraction.

The systems of the present embodiments can comprise, independently and optionally, means for gathering fishes from which lipids are extracted (e.g., nets), means for conveying the gathered fishes from the fish enclosure or a holding enclosure to the fish processing facility (e.g., pipes, conveyors, bins, trucks), means for cutting large pieces of fish into small pieces before cooking and pressing (e.g., chopper, hogger), means for heating the fishes to about 70° C., 80° C., 90° C. or 100° C. (e.g., steam cooker); means for grinding, pureeing, and/or pressing the fishes to obtain lipids (e.g., single screw press, twin screw press, with capacity of about 1-20 tons per hour); means for separating lipids from the coagulate (e.g., decanters and/or centrifuges); means for separating the oily phase from the aqueous fraction (e.g., decanters and/or centrifuges); and means for polishing the lipids (e.g, reactor for transesterification or hydrogenation). Many commercially available systems for producing fish meal can be adapted for use in the embodiments provided herein, including stationary and mobile systems that are mounted on a container frame or a flat rack. The fish oil, or a composition comprising fish lipids, can be collected and used as an EPA and/or DHA-rich oil, or upgraded to an EPA and/or DHA concentrate.

5.4 EPA, DHA, and Other Lipids

The present embodiments provide an EPA and/or DHA feedstock or an EPA and/or DHA comprising lipids, hydrocarbons, or both, derived from fish that harvested algae according to the methods provided herein. Lipids of the present embodiments can be subdivided according to polarity: neutral lipids and polar lipids. The major neutral lipids are triglycerides, and free saturated and unsaturated fatty acids. The major polar lipids are acyl lipids, such as glycolipids and phospholipids. A composition comprising lipids and hydrocarbons of the present embodiments can be described and distinguished by the types and relative amounts of key fatty acids and/or hydrocarbons present in the composition.

Fatty acids are identified herein by a first number that indicates the number of carbon atoms, and a second number that is the number of double bonds, with the option of indicating the position of the first double bond or the double bonds in parenthesis. The carboxylic group is carbon atom 1 and the position of the double bond is specified by the lower numbered carbon atom. For example, EPA is identified as 20:5 (n-3), which is all-cis-5,8,11,14,17-eicosapentaenoic acid, and DHA is identified as 22:6 (n-3), which is all-cis-4,7,10,13,16,19-docosahexaenoic acid, or DHA. The n-3 designates the location of the double bond, counting from the end carbon (highest number).

Algae produce mostly even-numbered straight chain saturated fatty acids (e.g., 12:0, 14:0, 16:0, 18:0, 20:0 and 22:0) with smaller amounts of odd-numbered acids (e.g., 13:0, 15:0, 17:0, 19:0, and 21:0), and some branched chain (iso- and anteiso-) fatty acids. A great variety of unsaturated or polyunsaturated fatty acids are produced by algae, mostly with C₁₂ to C₂₂ carbon chains and 1 to 6 double bonds, mainly in cis configurations. Fatty acids produced by the cultured algae of the present embodiments comprise one or more of the following: 12:0, 14:0, 14:1, 15:0, 16:0, 16:1, 16:2, 16:3, 16:4, 17:0, 18:0, 18:1, 18:2, 18:3, 18:4, 19:0, 20:0, 20:1, 20:2, 20:3, 20:4, 20:5, 22:0, 22:5, 22:6, and 28:1 and in particular, 18:1(9), 18:2(9,12), 18:3(6, 9, 12), 18:3(9, 12, 15), 18:4(6, 9, 12, 15), 18:5(3, 6, 9, 12, 15), 20:3(8, 11, 14), 20:4(5, 8, 11, 14), 20:5(5, 8, 11, 14, 17), 20:5(4, 7, 10, 13, 16), 20:5(7, 10, 13, 16, 19), 22:5(7, 10, 13, 16, 19), 22:6(4, 7, 10, 13, 16, 19). Without limitation, it is expected that many of these fatty acids are present in the lipids extracted from the fishes that ingested the cultured algae.

The hydrocarbons present in algae are mostly straight chain alkanes and alkenes, and may include paraffins and the like having up to 36 carbon atoms. The hydrocarbons are identified by the same system of naming carbon atoms and double bonds as described above for fatty acids. Non-limiting examples of the hydrocarbons are 8:0, 9,0, 10:0, 11:0, 12:0, 13:0, 14:0, 15:0, 15:1, 15:2, 17:0, 18:0, 19:0, 20:0, 21:0, 21:6, 23:0, 24:0, 27:0, 27:2(1, 18), 29:0, 29:2(1, 20), 31:2(1,22), 34:1, and 36:0.

A great variety of unsaturated or polyunsaturated fatty acids are produced by fish mostly with C₁₂ to C₂₂ carbon chains and 1 to 6 double bonds, mainly in cis configurations (Stansby, M. E., “Fish oils,” The Avi Publishing Company, Westport, Conn., 1967). Fish oil comprises about 90% triglycerides, about 5-10% monoglycerides and diglycerides, and about 1-2% sterols, glyceryl ethers, hydrocarbons, and fatty alcohols. One of skill would understand that the amount and variety of lipids in fish oil varies from one fish species to another, and also with the season of the year, the algae diet, spawning state, and environmental conditions. Fatty acids produced by the fishes of the present embodiments comprise, without limitation, one or more of the following: 12:0, 14:0, 14:1, 15:branched, 15:0, 16:0, 16:1, 16:2 n-7, 16:2 n-4, 16:3 n-4, 16:3 n-3, 16:4 n-4, 16:4 n-1, 17:branched, 17:0, 17:1, 18:branched, 18:0, 18:1, 18:2 n-9, 18:2 n-6, 18:2 n-4, 18:3 n-6, 18:3 n-6, 18:3 n-3, 18:4 n-3, 19:branched, 19:0, 19:1, 20:0, 20:1, 20:2 n-9, 20:2 n-6, 20:3 n-6, 20:3 n-3, 20:4 n-6, 20:4 n-3, 20:5 n-3, 21:0, 21:5 n-2, 22:0, 22:1 n-11, 22:2, 22:3 n-3, 22:4 n-3, 22:5 n-3, 22:6 n-3, 23:0, 24:0, 24:1 (where n is the first double bond counted from the methyl group). See, also Jean Guillaume, Sadisivam Kaushik, Pierre Bergot, and Robert Metailler, “Nutrition and Feeding of Fish and Crustaceans,” Springer-Praxis, UK, 2001).

In certain embodiments, EPA and/or DHA in the predominant form of triglyceride esters can be converted to lower alkyl esters, such as methyl, ethyl, or propyl esters, by known methods and used in an esterification with a sterol to form esters, which can be further purified for use as nutritional supplement. Transesterification, in general, is well known in the art. See, e.g., W. W. Christie, “Preparation of Ester Derivatives of Fatty Acids for Chromatographic Analysis,” Advances in Lipid Methodology—Volume Two, Ch. 2, pp. 70-82 (W. W. Christie, ed., The Oily Press, Dundee, United Kingdom, 1993).

In certain embodiments, to obtain a refined product with higher concentrations of EPA and/or DHA, certain lipases can be used to selectively transesterify the ester moieties of EPA and/or DHA in fish oil triglycerides, under substantially anhydrous reaction conditions, as described in U.S. Pat. No. 5,945,318.

In certain embodiments, one or more edible additives can be included for consumption with the nutritional supplement of containing EPA and/or DHA. In one embodiment, additives can include one or more antioxidants, such as, vitamin C, vitamin E or rosemary extract. In one embodiment, additives can include one or more suitable dispersant, such as, lecithin, an alkyl polyglycoside, polysorbate 80 or sodium lauryl sulfate. In one embodiment, additives can include a suitable antimicrobial is, for example, sodium sulfite or sodium benzoate. In one embodiment, additives can include one or more suitable solubilizing agent is, such as, a vegetable oil such as sunflower oil, coconut oil, and the like, or mono-, di- or tri-glycerides.

In certain embodiments, additives can include, but not limited to, vitamins such as vitamin A (retinol, retinyl palmitate or retinol acetate), vitamin B1 (thiamin, thiamin hydrochloride or thiamin mononitrate), vitamin B2 (riboflavin), vitamin B3 (niacin, nicotinic acid or niacinamide), vitamin B5 (pantothenic acid, calcium pantothenate, d-panthenol or d-calcium pantothenate), vitamin B6 (pyridoxine, pyridoxal, pyridoxamine or pyridoxine hydrochloride), vitamin B 12 (cobalamin or cyanocobalamin), folic acid, folate, folacin, vitamin H (biotin), vitamin C (ascorbic acid, sodium ascorbate, calcium ascorbate or ascorbyl palmitate), vitamin D (cholecalciferol, calciferol or ergocalciferol), vitamin E (d-alpha-tocopherol, or d-alpha tocopheryl acetate) or vitamin K (phylloquinone or phytonadione).

In certain embodiments, additives can include, but not limited to, minerals such as boron (sodium tetraborate decahydrate), calcium (calcium carbonate, calcium caseinate, calcium citrate, calcium gluconate, calcium lactate, calcium phosphate, dibasic calcium phosphate or tribasic calcium phosphate), chromium (GTF chromium from yeast, chromium acetate, chromium chloride, chromium trichloride and chromium picolinate) copper (copper gluconate or copper sulfate), fluorine (fluoride and calcium fluoride), iodine (potassium iodide), iron (ferrous fumarate, ferrous gluconate gluconate, magnesium hydroxide or magnesium oxide), manganese (manganese gluconate and manganese sulfate), molybdenum (sodium molybdate), phosphorus (dibasic calcium phosphate, sodium phosphate), potassium (potassium aspartate, potassium citrate, potassium chloride or potassium gluconate), selenium (sodium selenite or selenium from yeast), silicon (sodium metasilicate), sodium (sodium chloride), strontium, vanadium (vanadium surface) and zinc (zinc acetate, zinc citrate, zinc gluconate or zinc sulfate).

In certain embodiments, additives can include, but not limited to, amino acids, peptides, and related molecules such as alanine, arginine, asparagine, aspartic acid, carnitine, citrulline, cysteine, cystine, dimethylglycine, gamma-aminobutyric acid, glutamic acid, glutamine, glutathione, glycine, histidine, isoleucine, leucine, lysine, methionine, ornithine, phenylalanine, proline, serine, taurine, threonine, tryptophan, tyrosine and valine.

In certain embodiments, additives can include animal extracts such as cod liver oil, marine lipids, shark cartilage, oyster shell, bee pollen and d-glucosamine sulfate.

In certain embodiments, additives can include, but not limited to, unsaturated free fatty acids such as .gamma.-linoleic, arachidonic and .alpha.-linolenic acid, which may be in an ester (e.g., ethyl ester or triglyceride) form.

In certain embodiments, additives can include, but not limited to, herbs and plant extracts such as kelp, pectin, Spirulina, fiber, lecithin, wheat germ oil, safflower seed oil, flax seed, evening primrose, borage oil, blackcurrant, pumpkin seed oil, grape extract, grape seed extract, bark extract, pine bark extract, French maritime pine bark extract, muira puama extract, fennel seed extract, dong quai extract, chaste tree berry extract, alfalfa, saw palmetto berry extract, green tea extracts, angelica, catnip, cayenne, comfrey, garlic, ginger, ginseng, goldenseal, juniper berries, licorice, olive oil, parsley, peppermint, rosemary extract, valerian, white willow, yellow dock and yerba mate.

In certain embodiments, additives can include, but not limited to, enzymes such as amylase, protease, lipase and papain as well as miscellaneous substances such as menaquinone, choline (choline bitartrate), inositol, carotenoids (beta-carotene, alpha-carotene, zeaxanthin, cryptoxanthin or lutein), para-aminobenzoic acid, betaine HCl, free omega-3 fatty acids and their esters, thiotic acid (alpha-lipoic acid), 1,2-dithiolane-3-pentanoic acid, 1,2-dithiolane-3-valeric acid, alkyl polyglycosides, polysorbate 80, sodium lauryl sulfate, flavanoids, flavanones, flavones, flavonols, isoflavones, proanthocyanidins, oligomeric proanthocyanidins, vitamin A aldehyde, a mixture of the components of vitamin A₂, the D Vitamins (D₁, D₂, D₃ and D₄) which can be treated as a mixture, ascorbyl palmitate and vitamin K₂.

In certain embodiments, provided herein are liquid fuel compositions comprising EPA and/or DHA prepared from lipids extracted from fish that are controllably fed with algae according to the methods provided herein.

In certain embodiments, provided herein are liquid fuel compositions comprising mostly lipids devoid of EPA and/or DHA that have previously been separated from the crude fish lipids originally extracted from fish that are controllably fed with algae according to the methods provided herein.

The present invention may be better understood by reference to the following non-limiting examples, which are provided only as exemplary of the invention. The following examples are presented to more fully illustrate the preferred embodiments of the invention. The examples should in no way be construed, however, as limiting the broader scope of the invention.

### 6. Examples

6.1 Exemplary System

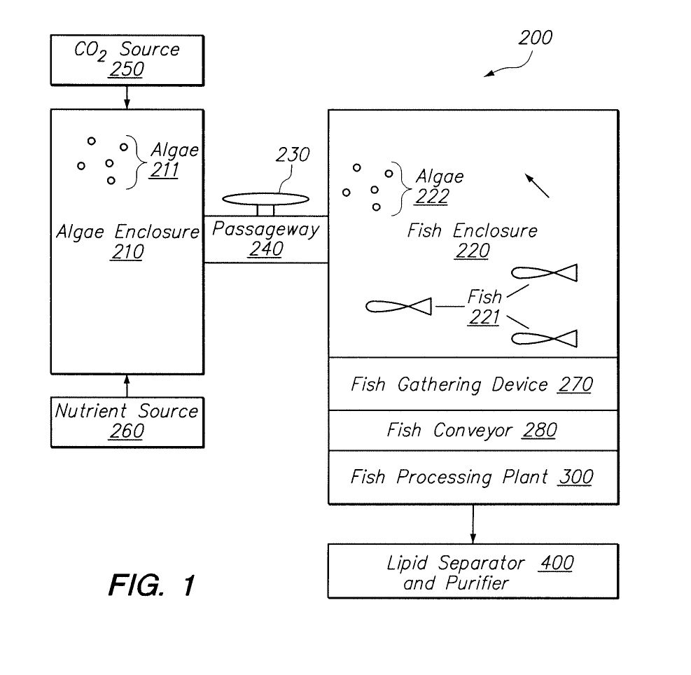

An overview of a method 100 of obtaining EPA and/or DHA, fishmeal, and/or high-grade fish fillets from fish, according to some embodiments of the invention, is described below and in FIG. 1. Referring to FIG. 1, first, an environment, an aquatic enclosure, a species of fish, and a consortium of algae species are selected to form a multi-trophic system 110 that produces EPA and/or DHA, fishmeal, and/or high-grade fish fillets efficiently. The environment and type of aquatic enclosure to be established in that environment are chosen to be hospitable to growth of the species of fish and algae. The species of algae and/or fish can be indigenous to the selected environment. The environment is preferably a parcel of non-arable land, which would avoid using land that could otherwise be used for food crops. The selected type of aquatic enclosure is then established in the selected environment 120.

A plurality of fish of the selected species and an algal composition comprising the selected species of algae are then introduced into the fish enclosure 130. The size of the populations is based, in part, on the size and characteristics of the enclosure and the growth characteristics of the particular species. After the initial addition of nutrients, the corresponding algal bloom takes 3-14 days. When the concentration of algae reaches the equivalent of 0.1 to 0.4 g/L, fish fingerlings are introduced to the pond. The number of fish depends on the species, but varies between 1,000 and 10,000 fingerlings per acre of pond.

Several physical parameters are closely monitored and if necessary controlled: pH, nitrogen concentration, phosphorus concentration, salinity, temperature, pH, O2, algae concentration, composition of the algae population, fish number, and fish size and weight. While the productivity of fish is the primary metric for the system, the consistent production of algae and fish biomass is a critical component. pH is controlled by bubbling or sparging CO₂ into the ponds, or adding weak acids (e.g., carbonic acid), bases, or buffers (sodium bicarbonate), which provides more precise control. The nutrient levels are controlled by adding water to the ponds (diluting), reducing discharge (accumulating), or adding fertilizer, as needed. Salinity is adjusted by either adding more water (dilution), decreasing discharge (accumulation), or evaporative spraying (concentration). Temperature is controlled through evaporative sprays for cooling. Oxygen levels are maintained through the use of aerators.

The algae in the system are exposed to light from the sun 140, which encourages growth of the algae. A majority portion of the algae is harvested with the population of fish 150. Usefully, the portion of algae that is not consumed can reproduce in the enclosure and thus replenish the algae population. In certain embodiments, a equilibrium can be maintained between the fish population and the algae that continue to grow in the fish enclosure.

After a predefined amount of time (e.g., after the fish grow to a specified size, or after the growth rate of the fish drops below a specified value), a plurality of fish are gathered 150, e.g., using conventional fishery techniques such as netting. Optionally, some fish are left in the enclosure to reproduce and thus replenish the fish population. In other embodiments, substantially all of the fish are gathered and processed for EPA and/or DHA 170. According to the invention, a new batch of fish of the selected species is introduced into the enclosure. The cycle of adding algae followed by algal growth 140, harvesting the algae 150, gathering the fish 160, conversion of the fish into EPA and/or DHA, fishmeal, high-grad fish fillets, 170, and introduction of a new batch of fish can be repeated as many times as desired, so long as the environment and aquatic enclosure remain suitable for growth of the fish population.

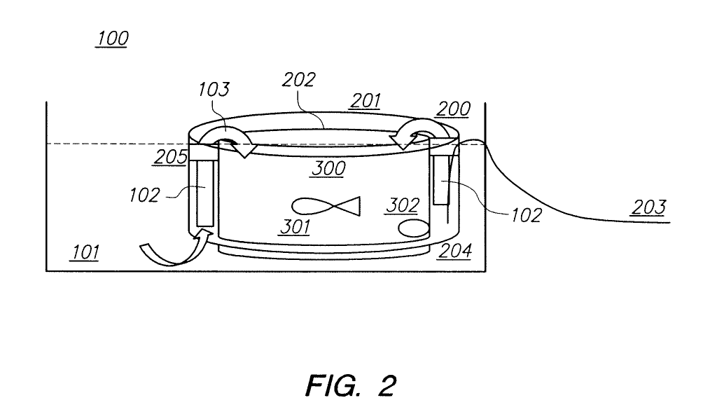

In another embodiment of the invention, the fishes and algae are grown separately from each other for at least part of the time before the fishes are allowed to harvest the algae. FIG. 2 illustrates a system 200 that grows the algae separately from the fishes. System 200 includes a growth enclosure 210, a fish enclosure 220, a gate 230, and an aquatic passageway 240 that provides fluidic transportation of algae from growth enclosure 210 to fish enclosure 220 when gate 230 is opened. Selected species of algae are introduced into the water in growth enclosure 210, which is connected to CO₂ source 250 and/or nutrient source 260. Because there are substantially no fish in growth enclosure 210, the growth of algae 211 is essentially unchecked. Then, after the algae 211 reaches a sufficient density, the gate 330 is opened and the algae flows through aquatic passageway 340 into fish enclosure 320. There, fishes 221 harvest algae 222 and grow to a desirable size or weight. After a period of growth, the fishes are gathered or harvested by device 270 and move by a conveyor 280 to fish processing plant 300 where EPA and/or DHA, fishmeal, high-grade fish fillets, and/or fish lipids are extracted. The fish lipids can be further separated and purified into two fractions: an EPA and DHA concentrate and non-EPA/DHA lipids in separator and purifier 400.

6.2 Menhaden Culture

In this example, at a laboratory scale, menhaden from the Gulf of Mexico were raised in indoor tanks to harvest cultured algae that are native to the Texas Gulf coast, and the results were used to initiate a pilot operation involving about 1000 menhaden in an open pond near Rio Hondo, Texas. Menhaden are generally abundant in the Gulf, and thus have never been cultured for aquaculture purpose.

One of the challenges of culturing phytoplanktivorous fish is providing algae that are sufficiently large for the fish to retain in its filter. Durbin and Durbin (1975, Grazing rates of the Atlantic menhaden I as a function of particle size and concentration, Marine Biol. 33:265-277) reported that the size threshold for the Atlantic menhaden (Brevoortia tyrannus) is 13-16 μm. Diatoms collected from ponds in Texas that can be cultured in the laboratory are typically smaller (<10 μm) with notable exceptions which include strains of the genus Amphiprora that is approximately 20 μm and Thalassiosira sp. which range in size between 8 and 15 μm. Other algae with spines (including Chaetoceros sp. and Cylindrotheca sp.) are able to be cleared by the menhaden, likely due to their increased surface area which results in a clumping effect. Other algae which are easily cultured in the lab are smaller in size individually but seem to connect in a filamentous manner, and would also be candidates for successful menhaden filtering.

In the laboratory tests, small cohorts (10-15 fish) of age 0 menhaden, including both species of Brevoortia gunteri (Gulf menhaden) and Brevoortia patronus (fine scale Gulf menhaden), were fed algae that are native to the Texas Gulf Coast, including strains of Isochrysis (4-6 μm in size), Chaetoceros (6-8 μm) and Amphiprora (20 μm). On average, the menhaden were approximately 65-70 mm in length and weighed about 5 g. Both species are indigenous to the Gulf Coast, phytoplanktivorous in its feeding habit, and efficient accumulators of lipids. The size of the algae was measured by a Beckman Coulter Counter (Multisizer-3). The algae were initially taken from ponds at Rio Hondo, isolated in a laboratory, and were then mass-cultured in 80-liter photobioreactors. The algae were selected for a combination of larger size, fast reproduction rate (doubling every 2-3 days), and higher lipid content (10-18%). The experiments were performed in duplicate in 140-liter tanks each containing 10-15 fish. Approximately 20 liters of green water (100-1000 mg/L algae from photobioreactors) were added to the tanks and the algae concentration was monitored with the Coulter Counter. A third tank was used as a control. The menhaden effectively filter-fed on the Chaetoceros (algae with spinesand corresponding increased surface area) and Amphiprora (20 micron-sized algae), clearing 80-100% of the algae over 24 hrs, and easily consumed 5-10% of their body weight daily in algae (dry weight basis). However, the menhaden did not consume the Isochrysis.

In the pilot operations, several 5-acre unlined ponds near Rio Hondo were prepared by removing existing large vegetation, such as bushes, and plowing into the ground smaller vegetation, such as grasses, weeds. The ground was tilled to allow bacterial decomposition of the biomass at the bottom of the ponds. The ponds were then filled with saline water. Water salinity at Rio Hondo varies seasonally from 10 to 17 parts per thousand depending mostly on rainfall. The water was coarsely screened by 1 mm filter to remove debris and aquatic organisms, such as fish. The native consortium of microalgae served as the initial inoculum for the ponds.

A mixed population of B. gunteri and B. patronus (<5% B. gunteri) comprising approximately three thousand menhaden (year 0) were cultured in five 5-acre ponds for forty weeks. The population of menhaden filter-fed on the natural algae bloom that was induced by inorganic fertilizers (urea and mono-ammonium phosphate) in one pond and organic fertilizers (pelletized fish feed via fish waste or uneaten feed) in the others. The fish grew from an average of 5 g to 120 g and from 70 mm to 170 mm in forty weeks with minimal mortality (<5% of population). The growth rate obtained is comparable to that of wild Gulf menhaden as reported in Vaughan, D. S., Smith, Joseph W., and Prager, Michael H. (2000), “Population Characteristics of Gulf Menhaden, Brevoortia patronus,” NOAA Technical Report NMFS 149. The menhaden lipid profile indicated a concentration of 16% EPA and DHA.

All references cited herein are incorporated herein by reference in their entirety and for all purposes to the same extent as if each individual publication or patent or patent application was specifically and individually indicated to be incorporated by reference in its entirety for all purposes.

Many modifications and variations of this invention can be made without departing from its spirit and scope, as will be apparent to those skilled in the art. The specific embodiments described herein are offered by way of example only, and the invention is to be limited only by the terms of the appended claims along with the full scope of equivalents to which such claims are entitled.

What is claimed is:

1. A method for producing an EPA and/or DHA-containing oil, said method comprising:
(i) growing one or more algae species that produce EPA and/or DHA in a first enclosed-container system or open-pond system;
(ii) harvesting said algae, comprising controllably feeding said algae to one or more zooplankton and/or fish species that feed on said algae in said first enclosed-container system or open-pond system, or in a second enclosed-container system or open-pond system, wherein said fish species feed on said algae and/or zooplankton; and
(iii) extracting EPA- and/or DHA-containing lipids from the fish, wherein said EPA- and/or DHA-containing lipids are processed to concentrate and purify EPA and/or DHA.

2. The method of claim 1, further comprising processing the lipids to form EPA- and/or DHA-containing products for human consumption or animal feeds.

3. The method of claim 2, wherein said extracting step comprises a processing technique selected from chromatography, fractional or molecular distillation, enzymatic splitting, low-temperature crystallization, supercritical fluid extraction, or urea complexation.

4. The method of claim 1, wherein said growing step comprises culturing said algae in a successive scale-up system comprising:
(i) inoculating said algae in a volume of 50 to 200 ml;
(ii) transferring said algae culture to a vessel, open pond, or raceway that is 2 to 20 times larger in volume when the algae concentration reaches 50 to 1000 mg/L, wherein said transferring step is repeated until a desired amount of algae is grown; and
(iii) harvesting the algae as a single batch or semi-batch wise, wherein a fraction of the algae is harvested daily and replaced with additional water and nutrients.

5. The method of claim 1, wherein the harvesting step comprises controllably feeding the algae to the fish while the fish is growing from fry to juvenile, from juvenile to adult, from fry to adult or to adult grow-out.

6. The method of claim 1, wherein the harvesting step comprises controllably feeding the algae to the fish until at least 50% of a dominant species within the population reaches a fish biomass set point, wherein the fish biomass set point is determined by mass, density, lipid content, or lipid profile.

7. The method of claim 1, wherein the harvesting step comprises feeding the algae to the fish according to an algal biomass set point, wherein the algal biomass set point is determined by mass, density, lipid content, or lipid profile.

8. The method of claim 1, wherein the harvesting step comprises gathering the fish that reaches a fish biomass set point, wherein the fish biomass set point is determined by mass, density, lipid content, or lipid profile.

9. The method of claim 1, wherein growth of said algae species is controlled by manipulation of one or more cultivation conditions selected from a group consisting of nutrient concentration, salinity, alkalinity, pH, temperature, carbon dioxide concentration, or mixing.

10. The method of claim 1, wherein the growing step and the harvesting step are carried out simultaneously in said first enclosed-container system or open-pond system.

11. The method of claim 10, wherein the fish feed on the algae continuously.

12. The method of claim 1, wherein the growing step and the harvesting step are carried out successively in said first enclosed-container system or open-pond system and said second enclosed-container system or open-pond system, respectively.

13. The method of claim 12, wherein the harvesting step comprises transferring a portion of a population of the fish or the entire population of fish at least once to said second enclosed-container system or open-pond system that has a lower loading density than said first enclosed-container system or open-pond system.

14. The method of claim 1, wherein the harvesting step further comprises restocking the system with the algae and/or the fish.

15. The method of claim 1, wherein the harvesting step further comprises increasing or decreasing the number of fish of one or more species according to an algal biomass set point, wherein the algal biomass set point is determined by mass, density, lipid content, or lipid profile.

16. The method of claim 1, wherein the harvesting step comprises feeding the fish in said first or said second enclosed-container system or open-pond system that comprises the algae at a concentration of 10 to 1000 mg/L.

17. The method of claim 1, wherein the extracting step comprises heating the fish to a temperature between 70° C. to 100° C., pressing the fish to release the lipids, and separating the lipids from an aqueous phase and/or a solid phase.

18. The method of claim 1, wherein the extracting step comprises preparing a fishmeal composition from the fish, treating the fishmeal composition with near-critical or supercritical water, and separating the lipids from an aqueous phase and/or a solid phase.

19. The method of claim 1, wherein the algae are both prokaryotic and eukaryotic.

20. The method of claim 1, wherein the algae are microalgae and comprise at least a species of Skeletonema, Cyanophyceae, Trichodesmium, Cryptosphaera, Coelastrum, Chlorosarcina, Micractinium, Porphyridium, Nostoc, Closterium, Elakatothrix, Cyanosarcina, Trachelamonas, Euglena, Phacus, Synechocystis, Oscillatoria, Lyngbya, Kirchneriella, Carteria, Cryptomonas, Chlamydamonas, Synechococcus, Crococcus, Anacystis, Calothrix, Planktothrix, Anabaena, Hymenomonas, Isochrysis, Pavlova, Monodus, Monallanthus, Platymonas, Amphiprora, Chaetoceros, Pyramimonas, Nannochloropsis, Gymnodinium, Alexandrium, Cochlodinium, Dinophysis, Gyrodinium, Prorocentrum, Chattonella, Heterosigma, Glyphodesmis, Synedra, Neidium, Pinnularia, Stauroneis, Papiliocellulus, Scolioneis, Fallacia, Surirella, Entomoneis, Auricula, Stephanodiscus, Chroococcus, Staurastrum, Netrium, Chlorella, Amphora, Cymbella, Thalassiosira, Cylindrotheca, Rhodamonas, Nannochloropsis, Nitzchia, Pseudonitzchia, Navicula, Craticula, Gyrosigma, Pleurosigma, Melosira, Cosnodiscus, Haematococcus, Botryococcus and/or Tetraselmis.

21. The method of claim 1, wherein the fish comprise at least one fish species in the order Clupiformes, Siluriformes, Cypriniformes, Mugiliformes, and/or Perciformes.

22. The method of claim 1, wherein the fish comprise menhadens, shads, herrings, sardines, hilsas, anchovies, catfish, carps, milkfish, paddlefish, shiners, and/or minnows.

23. The method of claim 1, wherein the algae and the fish are freshwater species, marine species, briny species, or species that live in brackish water.

24. The method of claim 6, wherein the fish biomass set point is the 2-week weight, 2-week length, 2-week body depth, 2-week fat content, 4-week weight, 4-week length, 4-week body depth, 4-week fat content, 8-week weight, 8-week length, 8-week body depth, 8-week fat content, 3-month weight, 3-month length, 3-month body depth, 3-month fat content, 6-month weight, 6-month length, 6-month body depth, or 6-month fat content.

25. The method of claim 7, wherein the algal biomass set point is the 2-week weight, 2-week length, 2-week body depth, 2-week fat content, 4-week weight, 4-week length, 4-week body depth, 4-week fat content, 8-week weight, 8-week length, 8-week body depth, 8-week fat content, 3-month weight, 3-month length, 3-month body depth, 3-month fat content, 6-month weight, 6-month length, 6-month body depth, or 6-month fat content.

26. A method for producing an EPA and/or DHA-containing oil from algae, said method comprising:
(i) providing a multi-trophic system comprising algae and a population of fish in a plurality of enclosures, wherein the enclosures are on land adjacent to a coast;
(ii) growing the algae in one or more of the plurality of enclosures;
(iii) harvesting the algae by controllably feeding the algae to the population of fish, wherein at least a portion of the population of fish grows from fry to adulthood;
(iv) gathering at least a portion of the population of fish;
(v) extracting lipids from the gathered fish; and
(vi) further separating and purifying the lipids to form EPA and/or DHA-containing products.

27. The method of claim 26, wherein the algae and the population of fish are indigenous to the coast.

28. The method of claim 26, wherein the algae and the population of fish are growing in one of the plurality of enclosures and the fish feed on the algae continuously.

29. A multi-trophic system for producing an EPA and/or DHA feedstock comprising:
algae and fish in a plurality of enclosures;
(ii) means for controllably feeding the algae to the fish; and
(iii) means for extracting lipids from the fish, wherein the lipids are used to produce EPA- and/or DHA-containing products.

30. The multi-trophic system of claim 29 further comprising means for processing the lipids to form EPA- and/or DHA-containing products.

31. A composition comprising lipids extracted from fish that are controllably fed with algae according to the method of claim 1.

32. A liquid fuel composition comprising EPA and/or DHA prepared from lipids extracted from fish that are controllably fed with algae according to the method of claim 1.

33. A liquid fuel composition comprising mostly lipids devoid of EPA and/or DHA that have previously been separated from the crude fish lipids originally extracted from fish that are controllably fed with algae according to the method of claim 1.
